(19) United States
(12) Patent Application Publication
(10) Pub. No.: US 2026/0011406 A1
(43) Pub. Date: Jan. 8, 2026

(54) METHODS AND SYSTEMS FOR DETERMINING CLONALITY OF SOMATIC SHORT VARIANTS

(71) Applicant: Foundation Medicine, Inc., Boston, MA (US)

(72) Inventors: Ethan S. SOKOL, Somerville, MA (US); Smruthy Krishnarayapuram SIVAKUMAR, Everett, MA (US)

(73) Assignee: Foundation Medicine, Inc., Boston, MA (US)

(21) Appl. No.: 19/136,711

(22) PCT Filed: Dec. 8, 2023
(86) PCT No.: PCT/US2023/083221

§ 371 Date: Jun. 6, 2025

Related U.S. Application Data
(60) Provisional application No. 63/431,464, filed on Dec. 9, 2022.
(60) Provisional application No. 63/462,723, filed on Apr. 28, 2023.

(51) Int. Cl. G16B 20/20; C12Q 1/6855; G16H 10/40; G16H 20/40

(52) U.S. Cl. CPC G16B 20/20; C12Q 1/6855; G16H 10/40; G16H 20/40

(57) ABSTRACT

Methods and systems for determining a clonal fraction are described. The methods may comprise, for example, receiving sequence read data associated with the sample from a subject; determining at least one somatic alteration based on the sequence read data; determining a tumor fraction for each somatic alteration of the at least one somatic alteration based on the sequence read data, such that a plurality of tumor fractions is obtained; determining a sample tumor fraction, the sample tumor fraction corresponding to a highest tumor fraction from the tumor fraction for each somatic alteration of the at least one somatic alterations; and determining a clonal fraction of a somatic alteration of the at least one somatic alteration based on the sample tumor fraction and the corresponding tumor fraction of the somatic alteration.

### CROSS-REFERENCE TO RELATED APPLICATIONS

This application claims the priority benefit of U.S. Provisional Patent Application Ser. No. 63/431,464, filed Dec. 9, 2022, and to U.S. Provisional Patent Application Ser. No. 63/462,723, filed Apr. 28, 2023, the contents of each of which are incorporated herein by reference in their entirety.

### FIELD OF DISCLOSURE

The present disclosure relates generally to methods and systems for analyzing genomic profiling data, and more specifically to methods and systems for determining a clonal fraction distinguishing between clonal and subclonal events in tumor cells.

### BACKGROUND

Assessing a patient's mutational landscape over time and space may assist in improved diagnostic and prognostic care for the patient. For example, understanding the composition of mutations and the evolving cancer genome during cancer initiation and progression may provide valuable information regarding metastasis, drug resistance, and immune response of a subject. As an example, EGFR alterations in lung cancers typically occur early as a cancer initiating event. In such an example, each tumor cell associated with the initiating event would typically be expected to have an EGFR-L858R alteration. Thus, the EGFR-L858R alteration may be an example of a clonal alteration—e.g., an alteration found in each of the tumor cells associated with the disease (i.e., lung cancer).

As the cancer progresses the tumor cells may acquire additional alterations, e.g., a natural evolution of the tumor, as a response to treatment, or due to other factors. These additional alterations may have occurred as a resistance response to a patient's treatment. But these additional alterations may not be present in each of the tumor cells, but rather a sub-set of the tumor cells. The additional alteration found in the second group of tumor cells may correspond to a subclonal alteration—where a subclonal alteration is found in a subset (e.g., a smaller fraction) of the tumor cells. For instance, a first group of the tumor cells may have just the EGFR-L858R alteration, while a second group of the tumor cells may have the EGFR-L858R alteration and the additional alteration (e.g., EGFR T790M or C797S, which are commonly associated with treatment resistance).

The clonality of a pathogenic alteration (e.g., fraction of tumor cells exhibiting an alteration, thereby whether an alteration is clonal or subclonal) may provide insight into the tumor development process, which can impact a patient's treatment response and survival outcome. For instance, due to the differences in when and how these alterations occur, understanding which alterations in a sample correspond to clonal alterations and subclonal alterations, how many alterations correspond to clonal and subclonal alterations, and how these alterations interact with each other may be useful in informing clinical decision making. Accordingly, it may be useful to identify the fraction of tumor cells harboring a pathogenic alteration (e.g., a clonal or subclonal alteration), and this may provide improved insights into targeted therapeutic strategies. For example, targeting a clonal alteration that is present in a majority of tumor cells is generally preferred from a therapeutic approach.

Conventional techniques to determine a clonal fraction typically rely on whole exome sequencing (WES) or whole genome sequencing (WGS) to distinguish initiating driver events. But these techniques are time and resource intensive. Smaller data sets associated with targeted, panel-based NGS may also be used to profile a sample and are commonly used in a clinical setting. These smaller data sets, however, may not provide sufficient information to understand tumor evolution and inform potential resistance mechanism identification across different tumor types using conventional analysis techniques. Accordingly, there is a need to accurately identify the clonal fraction of an alteration in a sample using panel-based NGS to provide improved insights into therapeutic strategies in clinical settings.

### BRIEF SUMMARY

The cancer genome of a patient evolves during cancer initiation and progression, the composition of mutations may also change resulting in varying degrees of tumor heterogeneity. For instance, an initiating mutational driver event may result in a somatic alteration in all tumor cells (e.g., a clonal alteration). As additional alteration events occur, e.g., in response to treatment or disease progression, these later alterations may be present in a subset of tumor cells. This difference in the alteration make-up of the tumor cells leads to tumor heterogeneity. Tumor heterogeneity influences several aspects of tumor evolution, including tumor initiation, progression, metastasis, drug resistance, and the immune response. In order to obtain a detailed view about the tumor development process, it may be useful to identify the fraction of tumor cells harboring a pathogenic mutation, which can be used to inform targeted therapeutic strategies.

Disclosed herein are methods and systems for determining a clonal fraction of a sample from a patient. Specifically, embodiments of the present disclosure may provide a more accurate representation of the fraction of tumor cells harboring an alteration of interest based on determining a clonality of the alteration, particularly when the determination is based on targeted and/or panel-based next-generation sequencing (NGS). For example, whole exome sequencing (WES) or whole genome sequencing (WGS) have been used to distinguish initiating driver events, these techniques are time and resource intensive. Smaller data sets associated with targeted, panel-based NGS may also be used to profile a sample. These smaller data sets, however, may not provide sufficient information to understand tumor evolution and inform potential resistance mechanism across different tumor types using conventional analysis techniques. Embodiments of the present disclosure aim to improve the understanding of tumor evolution using targeted, panel-based NGS by inferring a clonality of short variants (e.g., somatic alterations) in a single sample, as described herein.

In this manner, embodiments of the present disclosure provide techniques for producing an accurate clonality prediction and/or clonal fraction estimate. In some embodiments, the system can estimate a clonal fraction based on targeted, panel-based NGS. As discussed above, determining sample-wide estimates may be challenging based on panel-based NGS because the gene sequencing data corresponds to selected portions of the genome, as opposed to the entire genome as in WES or WGS. As a result, the data derived from panel-based NGS may not be as comprehensive as data derived from WES and WGS and using conventional analysis techniques (e.g., used to determine the clonal fraction based on WES and/or WGS) may provide inaccurate results when applied to data derived from panel-based. For instance, determining an accurate clonal fraction relies on locus copy number and mutant copy number. But models based on panel-based NGS data may not accurately model the locus copy number and mutant copy number. Another drawback to conventional methods for determining clonal fraction is that panel-based NGS is commonly used in a clinical setting. Thus, healthcare providers may not have access to data derived from WES or WGS techniques to obtain an accurate clonal fraction estimate. Accordingly, developing techniques for accurately analyzing panel-based NGS sequence data to determine a clonal fraction is more applicable in a clinical setting than analyses based on WES or WGS. Embodiments of the present disclosure overcome these challenges by providing improved techniques for determining a clonal fraction based on panel-based NGS sequence data. For instance, embodiments of the present disclosure select a proxy sample tumor fraction value based on a highest determined tumor fraction value from the somatic alterations identified in the sample (e.g., where the sample includes noncoding alterations and alterations in baited regions such as single nucleotide polymorphisms (SNPs) or regions next to baited exons). Accordingly, embodiments of the present disclosure can determine an accurate clonal fraction estimate and accurately predict clonality of one or more somatic alterations in a sample even when relying on sequence read data from panel-based NGS.

Embodiments of the present disclosure provide methods comprising: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; determining, using the one or more processors, at least one somatic alteration based on the sequence read data; determining, using the one or more processors, a tumor fraction for each somatic alteration from the at least one somatic alteration, such that at least one tumor fraction is obtained; identifying, using the one or more processors, a tumor fraction with a highest value from the at least one tumor fraction as a sample tumor fraction; and determining, using the one or more processors, a clonal fraction for each somatic alteration from the at least one somatic alteration based on (1) the sample tumor fraction, and (2) a corresponding tumor fraction for each somatic alteration.

In one or more embodiments, the corresponding tumor fraction is associated with a corresponding somatic alteration of the plurality of somatic alterations, the corresponding tumor fraction determined based on an allelic frequency of the corresponding somatic alteration, a mutant copy value of the corresponding somatic alteration, and a wildtype copy value of the corresponding somatic alteration. In one or more embodiments, one or more of the allelic frequency, the mutant copy value, or the wildtype copy value is based on the sequence read data.

In one or more embodiments, the method further comprises classifying, using the one or more processors, the corresponding somatic alteration of the sample as clonal if a corresponding clonal fraction is greater than a threshold. In one or more embodiments, the method further comprises classifying, using the one or more processors, the corresponding somatic alteration of the sample as subclonal if a corresponding clonal fraction is less than a threshold. In one or more embodiments, the threshold corresponds to a value of 0.5.

In one or more embodiments, the method further comprises determining whether a quality control (QC) metric of the sample exceeds a quality control threshold. In one or more embodiments, the QC metric is associated with one or more of: a sample purity; a sample noise; a sample aneuploidy; single-nucleotide polymorphism (SNP) data; an absence of somatic alterations; and an inconclusive copy number estimate.

In one or more embodiments, the subject is suspected of having or is determined to have cancer. In one or more embodiments, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In one or more embodiments, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In one or more embodiments, the method further comprises treating the subject with an anti-cancer therapy. In one or more embodiments, the anti-cancer therapy comprises a targeted anti-cancer therapy. In one or more embodiments, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In one or more embodiments, the method further comprises obtaining the sample from the subject. In one or more embodiments, the sample comprises a tissue biopsy sample, a liquid biopsy sample, or a normal control sample. In one or more embodiments, the sample is a liquid biopsy sample and comprises blood, plasma, cerebrospinal fluid, sputum, stool, urine, or saliva. In one or more embodiments, the sample is a liquid biopsy sample and comprises circulating tumor cells (CTCs). In one or more embodiments, the plurality of nucleic acid molecules comprises a mixture of tumor nucleic acid molecules and non-tumor nucleic acid molecules. In one or more embodiments, the sample is a liquid biopsy sample and comprises cell-free DNA (cfDNA), circulating tumor DNA (ctDNA), or any combination thereof. In one or more embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In one or more embodiments, the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In one or more embodiments, the one or more adapters comprise amplification primers, flow cell adaptor sequences, substrate adapter sequences, or sample index sequences. In one or more embodiments, the captured nucleic acid molecules are captured from the amplified nucleic acid molecules by hybridization to one or more bait molecules. In one or more embodiments, the one or more bait molecules comprise one or more nucleic acid molecules, each comprising a region that is complementary to a region of a captured nucleic acid molecule. In one or more embodiments, amplifying the one or more ligated nucleic acid molecules comprises performing a polymerase chain reaction (PCR) amplification technique, a non-PCR amplification technique, or an isothermal amplification technique.

In one or more embodiments, the sequencing comprises use of a massively parallel sequencing (MPS) technique, whole genome sequencing (WGS), whole exome sequencing, targeted sequencing, direct sequencing, or Sanger sequencing technique. In one or more embodiments, the sequencing comprises massively parallel sequencing, and the massively parallel sequencing technique comprises next generation sequencing (NGS). In one or more embodiments, the sequencer comprises a next generation sequencer.

In one or more embodiments, one or more of the plurality of sequencing reads overlap one or more gene loci within one or more subgenomic intervals in the sample. In such embodiments, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In one or more embodiments, the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCILI, WT1, XPO1, XRCC2, ZNF217, ZNF703, or any combination thereof.

In one or more embodiments, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In one or more embodiments, the method further comprises generating, by the one or more processors, a report indicating at least one clonal fraction of the clonal fraction for each somatic alteration from the at least one somatic alteration. In one or more embodiments, the method further comprises transmitting the report to a healthcare provider. In one or more embodiments, the report is transmitted via a computer network or a peer-to-peer connection.

Embodiments of the present disclosure further comprise methods for determining a clonal fraction associated with a sample from a subject. For instance, a method in accordance with embodiments of this disclosure can comprise: receiving, using one or more processors, sequence read data associated with the sample from the subject; determining, using the one or more processors, at least one somatic alteration based on the sequence read data; determining, using the one or more processors, a tumor fraction for each somatic alteration of the at least one somatic alteration based on the sequence read data, such that at least one tumor fraction is obtained; determining, using the one or more processors, a sample tumor fraction, the sample tumor fraction corresponding to a highest tumor fraction from the at least one tumor fraction; and determining, using the one or more processors, a clonal fraction of a somatic alteration of the at least one somatic alteration based on (1) the sample tumor fraction and (2) a corresponding tumor fraction of the somatic alteration.

In one or more embodiments, the corresponding tumor fraction of the somatic alteration is determined based on an allelic frequency of the somatic alteration, a mutant copy value of the somatic alteration, and a wildtype copy value of the somatic alteration. In one or more embodiments, one or more of the allelic frequency, the mutant copy value, or the wildtype copy value are obtained based on the sequence read data.

In one or more embodiments, the method further comprises classifying, using the one or more processors, the somatic alteration as clonal if the clonal fraction is greater than a threshold. In one or more embodiments, the method further comprises classifying, using the one or more processors, the somatic alteration as subclonal if the clonal fraction is less than the threshold. In one or more embodiments, the threshold corresponds to a value of 0.5.

In one or more embodiments, the method further comprises determining whether a quality metric (QC) of the sample exceeds a threshold. In one or more embodiments, the QC metric is associated with one or more of: a sample purity; a sample noise; a sample aneuploidy; single-nucleotide polymorphism (SNP) data; an absence of somatic alterations; and an inconclusive copy number estimate.

In one or more embodiments, the method further comprises in accordance with a determination that the clonal fraction is associated with a subclonal alteration, assigning, using the one or more processors, a therapy for the subject based on the clonal fraction. In one or more embodiments, the method further comprises in accordance with a determination that the clonal fraction is associated with a subclonal alteration, administering, using the one or more processors, a treatment to the subject based on the clonal fraction. In one or more embodiments, therapy comprises a targeted therapy.

In one or more embodiments, the method further comprises in accordance with a determination that the clonal fraction is associated with a clonal alteration, assigning, using the one or more processors, a treatment to the subject based on the clonal fraction, wherein the therapy comprises a therapy configured to target the clonal alteration. In one or more embodiments, the method further comprises determining, using the one or more processors, a prognosis of the subject based on the clonal fraction. In one or more embodiments, the method further comprises monitoring, using the one or more processors, a progression of a disease of the subject based on the clonal fraction. In such embodiments, the clonal fraction corresponding to a subclonal alteration is indicative of at least one of a poor prognosis, disease progression, and treatment resistance.

In one or more embodiments, the method further comprises in accordance with a determination that the clonal fraction is associated with a clonal alteration, identifying the respective alteration as a driver of disease in the subject. In one or more embodiments, the method further comprises predicting, using the one or more processors, one or more clinical outcomes based on the clonal fraction. In one or more embodiments, wherein the sequence read data for the subject is based on one or more of a broad panel sequencing panel, a targeted-exome sequencing panel, or a whole exome sequencing technique. In one or more embodiments, the method further comprises in accordance with a determination that the clonal fraction is associated with a subclonal alteration, recommending chemotherapy as a treatment.

In one or more embodiments, the sequence read data for the subject is derived from multiple biopsy samples or a single biopsy sample. In one or more embodiments, the sequence read data for the subject is derived from single cell sequencing. In one or more embodiments, the sequence read data for the subject is derived from circulating tumor DNA in a liquid biopsy sample.

In one or more embodiments, the determination of the clonal fraction is used to diagnose or confirm a diagnosis of disease in the subject. In one or more embodiments, the disease is cancer. In one or more embodiments, the cancer is at least one of is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In one or more embodiments, the method further comprises selecting an anti-cancer therapy to administer to the subject based on the determination of the clonal fraction. In one or more embodiments, the method further comprises determining an effective amount of an anti-cancer therapy to administer to the subject based on the determination of the clonal fraction. In one or more embodiments, the method further comprises administering the anti-cancer therapy to the subject based on the determination of the clonal fraction. In such embodiments, the anti-cancer therapy comprises chemotherapy, radiation therapy, immunotherapy, a targeted therapy, or surgery.

Embodiments of the present disclosure further comprise methods for diagnosing a disease, the methods comprising: diagnosing that a subject has the disease based on a determination of the clonal fraction for a sample from the subject, wherein the clonal fraction is determined according to any of the methods described above.

Embodiments of the present disclosure further comprise methods for selecting an anti-cancer therapy, the methods comprising: responsive to determining the clonal fraction for a sample from a subject, selecting an anti-cancer therapy for the subject, wherein the clonal fraction is determined according to according to any of the methods described above.

Embodiments of the present disclosure further comprise methods for treating a cancer in a subject, the methods comprising: responsive to determining the clonal fraction associated with a sample from the subject, administering an effective amount of an anti-cancer therapy to the subject, wherein the clonal fraction is determined according to according to any of the methods described above.

A method for monitoring cancer progression or recurrence in a subject, the method comprising: determining a first clonal fraction in a first sample obtained from the subject at a first time point according to according to any of the methods described above; and determining a second clonal fraction in a second sample obtained from the subject at a second time point; and comparing the first clonal fraction to the second clonal fraction, thereby monitoring the cancer progression or recurrence. In such methods, the second the clonal fraction for the second sample is determined according to according to any of the methods described above. In one or more embodiments, such methods further comprise selecting an anti-cancer therapy for the subject in response to the cancer progression. In one or more embodiments, such methods further comprise administering an anti-cancer therapy to the subject in response to the cancer progression. In one or more embodiments, such methods further comprise adjusting an anti-cancer therapy for the subject in response to the cancer progression. In one or more embodiments, such methods further comprise adjusting a dosage of the anti-cancer therapy or selecting a different anti-cancer therapy in response to the cancer progression. In one or more embodiments, such methods further comprise administering the adjusted anti-cancer therapy to the subject. In one or more embodiments, the first time point is before the subject has been administered an anti-cancer therapy, and wherein the second time point is after the subject has been administered the anti-cancer therapy. In one or more embodiments, the subject has a cancer, is at risk of having a cancer, is being routine tested for cancer, or is suspected of having a cancer. In one or more embodiments, the cancer is a solid tumor. In one or more embodiments, the cancer is a hematological cancer. In one or more embodiments, the anti-cancer therapy comprises chemotherapy, radiation therapy, immunotherapy, a targeted therapy, or surgery.

Methods disclosed above in accordance with embodiments of this disclosure may further comprise determining, identifying, or applying the value of the clonal fraction associated with the sample as a diagnostic value associated with the sample. Methods disclosed above in accordance with embodiments of this disclosure may further comprise generating a genomic profile for the subject based on the determination of the clonal fraction. In one or more embodiments, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In one or more embodiments, the genomic profile for the subject further comprises results from a nucleic acid sequencing-based test. In one or more embodiments, the methods may further comprise selecting an anti-cancer therapy, administering an anti-cancer therapy, or applying an anti-cancer therapy to the subject based on the generated genomic profile.

In one or more embodiments, the determination of the clonal fraction associated with the sample is used in making suggested treatment decisions for the subject. In one or more embodiments, the determination of the clonal fraction associated with the sample is used in applying or administering a treatment to the subject.

Methods in accordance with embodiments of this disclosure may further comprise: providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; determining, using the one or more processors, a quality control metric of the sample; determining, using the one or more processors, a plurality of tumor fraction estimates for each of a plurality of somatic alterations identified in the sample based on the sequence read data; determining, using the one or more processors, a sample tumor fraction, the sample tumor fraction corresponding to a highest tumor fraction estimate from the plurality of tumor fraction estimates; determining, using the one or more processors, a plurality of clonal fractions each clonal fraction corresponding to each of the plurality of somatic alterations; and classifying, using the one or more processors, the sample as clonal with respect to a particular somatic alteration of the plurality of somatic alterations if a corresponding clonal fraction is greater than a threshold.

Embodiments of this disclosure may further comprise methods for determining a clonality of alterations in a sample. For instance, such methods can comprise: receiving, at one or more processors, sequence read data for a plurality of sequence reads derived from the sample; determining, using the one or more processors, a plurality of tumor fractions for each of a plurality of somatic alterations based on the sequence read data; determining, using the one or more processors, a sample tumor fraction, the sample tumor fraction corresponding to a highest tumor fraction from the plurality of tumor fractions; determining, using the one or more processors, a plurality of clonal fractions associated with the plurality of somatic alterations, a clonal fraction of the plurality of clonal fractions based on the sample tumor fraction and a tumor fraction for a particular somatic alteration of the plurality of somatic alterations; and classifying, using the one or more processors, the sample as clonal with respect to the particular somatic alteration if the clonal fraction is greater than a threshold.

Embodiments of the present disclosure can further comprise systems. For instance, a system according to embodiments of this disclosure can comprise: one or more processors; and a memory communicatively coupled to the one or more processors and configured to store instructions. In one or more embodiments, the instructions, when executed by the one or more processors, cause the system to perform a method, the method comprising: receiving, using one or more processors, sequence read data associated with the sample from the subject; determining, using the one or more processors, at least one somatic alteration based on the sequence read data; determining, using the one or more processors, a tumor fraction for each somatic alteration of the at least one somatic alteration based on the sequence read data, such that a plurality of tumor fractions is obtained; determining, using the one or more processors, a sample tumor fraction, the sample tumor fraction corresponding to a highest tumor fraction from the tumor fraction for each somatic alteration of the at least one somatic alterations; and determining, using the one or more processors, a clonal fraction of a somatic alteration of the at least one somatic alteration based on the sample tumor fraction and the corresponding tumor fraction of the somatic alteration.

Embodiments of the present disclosure can further comprise non-transitory computer-readable storage mediums storing one or more programs, the one or more programs comprising instructions, which when executed by one or more processors of a system, cause the system to perform a method comprising: receiving, using one or more processors, sequence read data associated with the sample from the subject; determining, using the one or more processors, at least one somatic alteration based on the sequence read data; determining, using the one or more processors, a tumor fraction for each somatic alteration of the at least one somatic alteration based on the sequence read data, such that a plurality of tumor fractions is obtained; determining, using the one or more processors, a sample tumor fraction, the sample tumor fraction corresponding to a highest tumor fraction from the tumor fraction for each somatic alteration of the at least one somatic alterations; and determining, using the one or more processors, a clonal fraction of a somatic alteration of the at least one somatic alteration based on the sample tumor fraction and the corresponding tumor fraction of the somatic alteration.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process flow for determining a clonality of a somatic alteration in accordance with embodiments of the present disclosure.

FIG. 2 provides a non-limiting example of a process flow for determining a treatment recommendation in accordance with embodiments of the present disclosure.

FIG. 3 provides a non-limiting example of a process flow for distinguishing germline and somatic genomic alterations in accordance with embodiments of the present disclosure.

FIG. 4A provides a non-limiting example of a schematic depiction of a section of a patient's genome.

FIG. 4B provides a non-limiting example of a schematic depiction of a FIG. 3 is a schematic depiction of genomic segmentation.

FIG. 5 provides a non-limiting example of a process flow for distinguishing germline and somatic genomic alterations in accordance with embodiments of the present disclosure.

FIG. 6 provides a non-limiting example of a process flow for determining a clonal fraction of a somatic alteration in accordance with embodiments of the present disclosure.

FIG. 7 depicts an exemplary computing device or system in accordance with one embodiment of the present disclosure.

FIG. 8 depicts an exemplary computer system or computer network, in accordance with some instances of the systems described herein.

### DETAILED DESCRIPTION

Disclosed herein are methods and systems for determining a clonal fraction of a somatic alteration identified in a sample from a patient. The estimated clonal fraction may be indicative of the portion of tumor cells that include a particular short variant (e.g., somatic alteration). Embodiments of the present disclosure may provide a more accurate representation of the fraction of tumor cells harboring an alteration of interest based on determining a clonality of the alteration, particularly when the determination is based on targeted and/or panel-based next-generation sequencing (NGS). For example, whole exome sequencing (WES) or whole genome sequencing (WGS) techniques have been used to profile genetic material included in a sample and distinguish initiating driver events. These techniques, however, are time and resource intensive. Smaller data sets associated with targeted, panel-based NGS may also be used to profile a sample, and are more commonly used in a clinical setting. These smaller data sets, however, may not provide sufficient information determine a clonality of alterations in a sample when using conventional methods to determine clonal fraction and clonality of a sample. Thus, it has been difficult to understand tumor evolution and inform potential resistance mechanism across different tumor types based on data from panel-based NGS.

Embodiments of the present disclosure include system and methods for determining a fraction of tumor cells harboring a somatic alteration identified in a patient's sample, e.g., a clonal fraction. In one or more examples, the clonal fraction may be used to determine a clonality of the corresponding somatic alteration. In one or more examples, determining the clonal fraction may be based on an estimated tumor fraction of the patient's sample (e.g., sample tumor fraction). The sample tumor fraction can be determined based on a highest tumor fraction selected from a plurality of tumor fractions associated with each somatic alteration identified in the sample. In some embodiments, the sample tumor fraction and clonal fraction may be determined based on sequence read data from targeted, panel-based NGS. Accordingly, embodiments of the present disclosure may improve the understanding of tumor evolution using targeted, panel-based NGS by inferring a clonality of short variants in a single sample, as described herein.

For instance, methods in accordance with embodiments of the present disclosure may comprise: receiving, using one or more processors, sequence read data associated with the sample from the subject; determining, using the one or more processors, at least one somatic alteration based on the sequence read data; determining, using the one or more processors, a tumor fraction for each somatic alteration of the at least one somatic alteration based on the sequence read data to obtain at least one tumor fraction; determining, using the one or more processors, a sample tumor fraction, the sample tumor fraction corresponding to a highest tumor fraction from the at least one tumor fraction; determining, using the one or more processors, a clonal fraction of a somatic alteration of the at least one somatic alteration based on (1) the sample tumor fraction and (2) the corresponding tumor fraction of the somatic alteration.

In some instances, for example, methods are described that comprise providing a plurality of nucleic acid molecules obtained from a sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, at one or more processors, sequence read data for the plurality of sequence reads; determining, using the one or more processors, at least one somatic alteration based on the sequence read data; determining, using the one or more processors, a tumor fraction for each somatic alteration from the at least one somatic alteration to obtain at least one tumor fraction; identifying, using the one or more processors, a tumor fraction with a highest value from the at least one tumor fraction as a sample tumor fraction; and determining, using the one or more processors, a clonal fraction for each somatic alteration from the at least one somatic alteration based on (1) the sample tumor fraction, and (2) a corresponding tumor fraction for each somatic alteration.

In some instances, for example, methods are described that comprise receiving, at one or more processors, sequence read data for a plurality of sequence reads derived from the sample; determining, using the one or more processors, a plurality of tumor fractions for each of a plurality of somatic alterations based on the sequence read data; determining, using the one or more processors, a sample tumor fraction, the sample tumor fraction corresponding to a highest tumor fraction from the plurality of tumor fractions; determining, using the one or more processors, a plurality of clonal fractions for the plurality of somatic alterations, a clonal fraction of the plurality of clonal fractions based on the sample tumor fraction and a tumor fraction for a particular somatic alteration of the plurality of somatic alterations; and classifying, using the one or more processors, the sample as clonal with respect to the particular somatic alteration if the clonal fraction is greater than a threshold.

In this manner, embodiments of the present disclosure provide techniques for producing an accurate clonality prediction and/or clonal fraction estimate. In some embodiments, the system can estimate a clonal fraction based on targeted, panel-based NGS. As discussed above, determining sample-wide estimates may be challenging based on panel-based NGS because the gene sequencing data corresponds to selected portions of the genome, as opposed to the entire genome as in WES or WGS. As a result, the data derived from panel-based NGS may not be as comprehensive as data derived from WES and WGS and using conventional analysis techniques (e.g., to determine the clonal fraction based on WES and/or WGS) may provide inaccurate results when applied to data derived from panel-based. For instance, determining an accurate clonal fraction relies on locus copy number and mutant copy number. But models based on panel-based NGS data may not accurately model the locus copy number and mutant copy number. Another drawback to conventional methods for determining clonal fraction is that panel-based NGS is commonly used in a clinical setting. Thus, healthcare providers may not have access to data derived from WES or WGS techniques to obtain accurate clonal fraction estimates. Accordingly, developing techniques for accurately analyzing panel-based NGS sequence data to determine a clonal fraction is more applicable in a clinical setting than analyses based on WES or WGS. Embodiments of the present disclosure overcome these challenges by providing improved techniques for determining a clonal fraction based on panel-based NGS sequence data. For instance, embodiments of the present disclosure select a proxy sample tumor fraction value based on a highest determined tumor fraction value from the somatic alterations identified in the sample (e.g., where the sample includes noncoding alterations and alterations in baited regions such as single nucleotide polymorphisms (SNPs) or regions next to baited exons). Accordingly, embodiments of the present disclosure can determine an accurate clonal fraction estimate and accurately predict clonality of one or more somatic alterations in a sample even when relying on sequence read data from panel-based NGS.

In order to determine an accurate clonal fraction and clonality of somatic alterations in a sample using sequencing data obtained from panel-based NGS, embodiments of the present disclosure rely on the maximum tumor fraction of the identified somatic variants in a sample as a proxy for the sample tumor fraction. As discussed above, early mutation events or initiating driver events (e.g., such as an EGFR-L858R) are expected to be present in each of the tumor cells. In some embodiments, early events or initiating driver events may be expected to be present in a majority of the tumor cells. Thus, the highest tumor fraction selected from the tumor fractions for each of the somatic alterations identified in a sample can be indicative of an initiating driver event or an early mutation event, e.g., because the highest tumor fraction is the alteration present in the greatest portion of the somatic tumor cells. Thus, in accordance with embodiments of this disclosure, the highest tumor fraction may be a reliable approximation for the tumor fraction because each of the tumor cells are expected to contain the corresponding alteration. In this manner, embodiments of the present disclosure can provide an accurate estimate of a sample tumor fraction in the absence of data obtained via WGS or WES techniques, e.g., using data obtained via panel-based NGS.

Additionally, embodiments of the present disclosure may be used to make treatment and therapy recommendations for patients based on an estimated clonal fraction and/or clonality of somatic alterations in a sample. For instance, determining a clonal fraction and/or determining the clonality of short variants, e.g., somatic alterations can be helpful in understanding the evolution of the disease in a patient, which can inform treatment recommendations and decisions.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “clonal fraction” refers to a fraction of tumor cells harboring a potentially pathogenic alteration.

As used herein, the term “clonal mutation” refers to a one or more mutations shared by cells associated with the same disease (e.g., cancer).

As used herein, the term “sub-clonal mutation” refers to one or more mutations shared by a sub-set of cells associated with the same disease (e.g., cancer).

The section headings used herein are for organizational purposes only and are not to be construed as limiting the subject matter described.

### Methods for Determining a Clonal Fraction of a Somatic Alteration

One or more embodiments of the present disclosure provide methods for determining a fraction of tumor cells harboring a somatic alteration identified in a patient's sample, e.g., a clonal fraction. In one or more examples, the clonal fraction may be used to determine a clonality (e.g., clonal or subclonal status) of the corresponding somatic alteration. In one or more examples, determining the clonal fraction may be based on an estimate of a sample tumor fraction. In some embodiments, estimating the sample tumor fraction may be performed when determining the clonal fraction based on sequence read data from targeted, panel-based NGS. The sample tumor fraction can be determined based on a highest tumor fraction selected from a plurality of tumor fractions associated with corresponding somatic alterations identified in the sample. Accordingly, embodiments of the present disclosure may improve the understanding of tumor evolution using targeted, panel-based NGS by inferring a clonality of short variants in a single sample, as described herein.

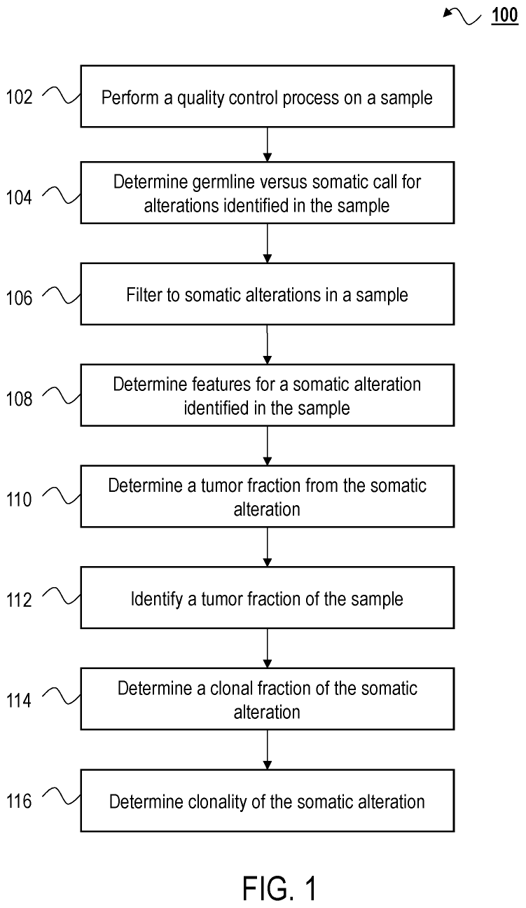

FIG. 1 provides a non-limiting example of a process 100 for determining a clonal fraction in accordance with embodiments of the present disclosure. Process 100 can be used for determining a clonal fraction of one or more short variants (e.g., somatic alterations) in a sample taken from an individual. In some examples, the process 100 can be used to determine a clonal fraction and/or estimate a clonality of a somatic alteration in the sample. In some instances, the sample may comprise a tissue biopsy sample. In some instances, the sample may be a liquid biopsy sample, and may comprise, e.g., blood, plasma, cerebrospinal fluid, sputum, stool, urine, or saliva.

In some instances, the sequence read data may be derived from sequencing of a single tissue biopsy sample collected from a single region of a tumor in a subject (e.g., a patient). In some instances, the sequence read data may be derived from sequencing a plurality of tissue biopsy samples collected from multiple regions of a tumor in the subject. In some instances, the sequence read data may be derived from a single cell sequencing method as opposed to a bulk tumor sequencing method. In some instances, the sequence read data may be derived from sequencing circulating tumor DNA in a liquid biopsy sample.

Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

As shown in FIG. 1, a sample may be quality controlled in Block 102. For example, certain conditions may be examined to determine if the sample is appropriate to be used to determine a clonal fraction and/or clonality of somatic alterations in a sample. During the quality control process, the system determines whether the sample meets minimum requirements associated with reliability for processing the sample. In one or more examples, if the sample fails to meet one or more quality control metrics, the system may not proceed to determine the clonal fraction for the sample. As discussed above, embodiments of the present disclosure may be performed using targeted sequencing and/or panel sequencing. Because the targeted sequencing provides a smaller data set compared to whole exome sequencing (WES) or whole genome sequencing (WGS), determining whether the sample meets one or more quality control (QC) metrics may ensure the accuracy of the clonal fraction determination.

In some instances, the system can determine whether the sample is reliable based on factors such as: a sample purity, a sample noise, a sample aneuploidy, single-nucleotide polymorphism (SNP) data; an absence of somatic alterations, and an inconclusive copy number estimate. In some embodiments, the sample may be determined to be reliable if it has a tumor purity of about 20-95%. In one or more examples, the sample may be determined to be reliable if the system determines the sample includes a characteristic aneuploidy event. The system may determine the sample is unreliable if the copy number profile of the sample is flat, which suggests issues with the copy number profile particularly in the absence of other clear driver mutations and/or fusions.

In some embodiments, the quality control process may further assess SNP data features, including but not limited to: an average sequence coverage for the sample, a minimum average sequence coverage for the sample, an allele coverage at each of the corresponding loci in the plurality of loci, a minimum allele coverage at each of the corresponding loci in the plurality of loci, a degree of nucleic acid contamination in the sample (determined, e.g., by quantifying aberrations in SNP allele frequencies), a maximum degree of nucleic acid contamination in the sample, a number of single nucleotide polymorphism (SNP) loci within the plurality of loci examined a minimum number of single nucleotide polymorphism (SNP) loci within the plurality of loci examined, or any combination thereof. Each SNP data features may be associated with a different quality control threshold. In some embodiments, the quality control threshold for the SNP data features may be predetermined. In some embodiments, the quality control thresholds may be determined empirically.

In some embodiments, if a sample is determined not to comprise somatic alterations (e.g., potentially pathogenic alterations), the system may not further process the sample. In some embodiments, the system may determine an inconclusive copy number estimate if the system determines that a curator has manually switched a copy number model; in such instances there may be challenges in accurately determining the copy number model for the sample. A copy number model may be switched if there was another model that better explained the plurality of data. Accordingly, such samples may be excluded from downstream analyses to determine the clonal fraction.

If the sample passes the quality control metrics in Block 102, the clonality may begin to be assessed. If the sample does not pass quality control, the clonality of the sample is not assessed. It will be understood that other quality control mechanisms may be implemented in addition, or alternative to, those described in Block 102. Alternatively, the sample may not pass quality control measures in certain instances.

At Block 104, the system can determine if an alteration is somatic or germline. For instance, a sample obtained from a patient is expected to include one or more alterations. The system may obtain sequence read data for the sample and identify one or more alterations based on the sequence read data. In some embodiments the alterations may be coding and/or non-coding alterations. Once the alterations are identified, the system can determine whether the each of the one or more alterations identified in the sample are somatic or germline, at Block 104. The process for determining whether an alteration is somatic or germline is described in more detail in the Methods for Distinguishing Somatic and Germline Alterations section, below. As an example, based on the analysis at Block 104, the system may determine that a sample has four somatic alterations (VS1, VS2, VS3, VS4) and one germline alteration (VG1).

At Block 106, the system can filter the alterations identified at Block 104. For instance, if the system determines that an alteration is germline, the system may not further process data regarding that alteration, and the clonal fraction and clonality for that germline alteration may not be determined. If the alteration is somatic, the system may further process data regarding that alteration to estimate the clonal fraction and clonality for the somatic alteration. Referring back to the example, the system can include the somatic alterations (VS1, VS2, VS3, VS4) in the downstream analysis and exclude the germline alteration (VG1). In some embodiments, the system may additionally filter alterations predicted to be associated with clonal hematopoiesis (CH). For instance, the system may not process alterations predicted to be CH-derived. In some instances, the system may not be able to determine whether a somatic alteration is somatic or germline (e.g., an inconclusive alteration-type). In such cases, the system may not process alterations associated with an inconclusive alteration-type result.

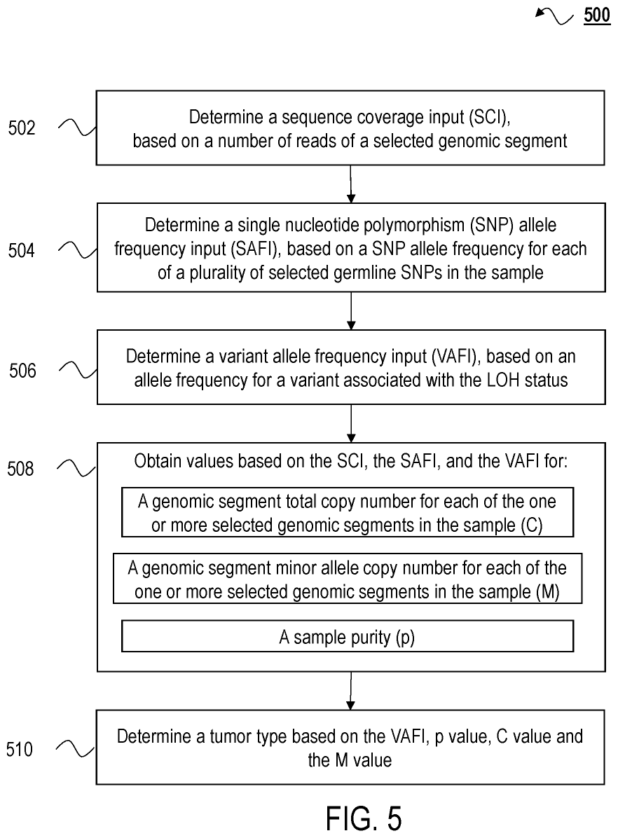

At Block 108, the system can determine one or more features associated with each of the somatic alterations identified at Block 106. For instance, for a given somatic alteration, the system may obtain an estimated allele frequency (AF) for the somatic alteration, a mutant allele copy (me) value for the somatic alteration and a reference copy (wc) value for the somatic alteration. In one or more examples, these values may be determined based on the values calculated or obtained at Block 104 as a part of the process for distinguishing somatic and germline alterations. See, e.g., FIG. 5, process 500, described in greater detail below. Referring again to the example, the system can obtain the variant allele frequency, mutant copy value and wild type copy value for each of VS1, VS2, VS3, VS4.

In some embodiments, an allele frequency (AF), can be determined based on the values obtained during process 500. As described above the me value, we value, and p value may be obtained and/or calculated as a part of a process for determining the tumor type. For instance, assuming a normal copy number value (e.g., copy number value of 2), the AFᵢ for a somatic alteration may be determined according to Equation 1:

$${AF}_{i} = \frac{VTF ( mc )}{VTF ( wc ) + VTF ( mc ) + 2 ( 1 - VF )} \qquad ( 1 )$$

where VTF is the tumor fraction for the short variant (e.g., somatic alteration), me is the mutant allele copy value, and we is the wild type copy value. As discussed above, because the AF value, mc value, and we values are determined during Block 104 as a part of determining whether an alteration is germline or somatic, Equation 1 can be rearranged to determine the tumor fraction for the somatic alteration, as shown in Equation 2, below:

$${VTF}_{i} = \frac{2 {AF}_{i}}{{mc}_{i} - {AF}_{i} ( {wc}_{i} + {mc}_{i} - 2 )} \qquad ( 2 )$$

At Block 110, the system can use the me value, we value, and AF determined by the system (e.g., at Block 104) to estimate or determine the tumor fraction for a somatic alteration identified in the sample. That is, the tumor fraction (VTF) for a specific somatic alteration may be based on the allele frequency, the mutant allele copy value, and the reference copy value of the somatic alteration. The tumor fraction estimation in Block 110 can be performed for one or more of the somatic alterations filtered at Block 106. In some embodiments, the tumor fraction can be estimated for each of the somatic alterations identified in Block 106. Referring back to the example, the tumor fraction can be estimated for each somatic variant as follows: VTF(VS1), VTF(VS2), VTF(VS3), and VTF(VS4).

At Block 112, the system can estimate a sample tumor fraction. The sample tumor fraction may be indicative of the fraction of cells that corresponds to tumor cells in the sample. In one or more embodiments, the highest tumor fraction (TFₘₐₓ) from the tumor fractions VTFᵢ (e.g., tumor fractions determined in Block 110) for the somatic alterations identified in a sample, is set to the tumor fraction (TF) estimate for the sample according to Equation 3:

$$TF = max ( {VTF}_{1} , {VTF}_{2} … , {VTF}_{i} ) \qquad ( 3 )$$

Referring back to the example, say VTF(VS1) 0.65, VTF(VS2) 0.54, VTF(VS3) 0.02, and VTF(VS4)=0.80. Based on these estimates VTF(VS4) would be set to the sample tumor fraction (e.g., TF=VTF(VS4)).

Determining a sample tumor fraction may be challenging, particularly for data derived from targeted, panel-based NGS. This is because, unlike whole genome sequencing (WGS) or whole exome sequencing (WES), which sequence the entire genome, panel-based NGS selects segments of the genome for analysis. As a result, the data derived from panel-based NGS may not be as comprehensive as data derived from WES and WGS. In instances where the clonal fraction and clonality are determined based on sequencing data obtained from panel-based NGS, embodiments of the present disclosure rely on the maximum tumor fraction of the identified somatic variants as a proxy for the sample tumor fraction.

As discussed above, early mutation events or initiating driver events (e.g., such as an EGFR-L858R) are expected to be present in each of the tumor cells. In some embodiments, early events or initiating driver events may be expected to be present in a majority of the tumor cells. Thus, the highest tumor fraction selected from the tumor fractions for each of the somatic alterations identified in a sample can be indicative of an initiating driver event or an early mutation event, e.g., because the highest tumor fraction is the alteration present in the greatest portion of the somatic tumor cells. Thus, the highest tumor fraction may be a reliable approximation for the tumor fraction because each of the tumor cells are expected to contain the corresponding alteration. In this manner, embodiments of the present disclosure can provide an accurate estimate of a sample tumor fraction in the absence of data obtained via WGS or WES techniques.

In Block 114, a clonal fraction for each somatic alteration may be determined as a ratio of the variant-estimated tumor fraction (VTF) to the sample-estimated tumor fraction (TF). For example, a clonal fraction may be determined based on Equation 4:

$$Clonal {fraction}_{i} = \frac{{VTF}_{i}}{TF} \qquad ( 4 )$$

Referring again to the example, Using Equation 4, the clonal fraction for VS1=0.65/0.80=0.81; VS2=0.54/0.80=0.66, VS3=0.02/0.80=0.03, VS4=0.80/0.80=1.00.

In some embodiments, a clonality of a somatic alteration of the sample may be determined in Block 116. For example, the somatic alteration may be determined to be clonal if the corresponding clonal fraction is greater than or equal to a threshold. In some instances, the threshold may be approximately 0.5. The sample may be subclonal if the clonal fraction is less than the threshold. In some instances, the threshold may be a different threshold, or may be a range of thresholds. Referring to the example, with a threshold of 0.5, VS1 (clonal fraction=0.81), VS2 (clonal fraction=0.66), and VS4 (clonal fraction=1.00) would be classified as clonal, while VS3 (clonal fraction=0.03) would be classified as subclonal.

In one or more examples, early events, such as EGFR-L858R would be expected to have a higher clonal fraction. As an example, an EGFR-L858R somatic alteration identified in a sample may be determined to have a clonal fraction of 0.7. Due to the early occurrence in the disease progression alterations associated with early events would be expected to be found in a majority of tumor cells and be associated with higher (e.g., greater than 0.5) clonal fraction values. Later events, e.g., mutation events that occur as resistance to treatment and/or natural disease progression are expected to be associated with a lower clonal fraction value, indicative that a smaller portion of the tumor cells include these alterations. As an example, an EGFR C797S somatic alteration identified in a sample may be determined to have a clonal fraction of 0.06 and/or a KRAS G12C somatic alteration identified in a sample may be determined to have a clonal fraction of 0.07. Accordingly, distinguishing clonal from subclonal alterations may be useful in evaluating a patient's mutational landscape. Based on the clonality of the sample, a patient's mutational landscape may be more accurately assessed, which may provide improved prognostic, diagnostic, and/or therapeutic options for treating the patient.

Accordingly, embodiments of the present disclosure provide techniques for producing an accurate clonality prediction and/or clonal fraction estimate. The estimated clonal fraction may be indicative of the portion of tumor cells that comprise a particular short variant (e.g., somatic alteration). In some embodiments, the system can estimate a clonal fraction based on targeted, panel-based NGS. As discussed above, determining sample-wide estimates may be challenging based on panel-based NGS because the gene sequencing data corresponds to selected portions of the genome, as opposed to the entire genome as in WES or WGS. Embodiments of the present disclosure overcome these challenges by selecting a proxy sample tumor fraction value based on a highest determined tumor fraction value from the somatic alterations identified in the sample. Accordingly, embodiments of the present disclosure can determine an accurate clonal fraction estimate and accurately predict clonality of one or more somatic alterations in a sample even when relying on sequence read data from panel-based NGS.

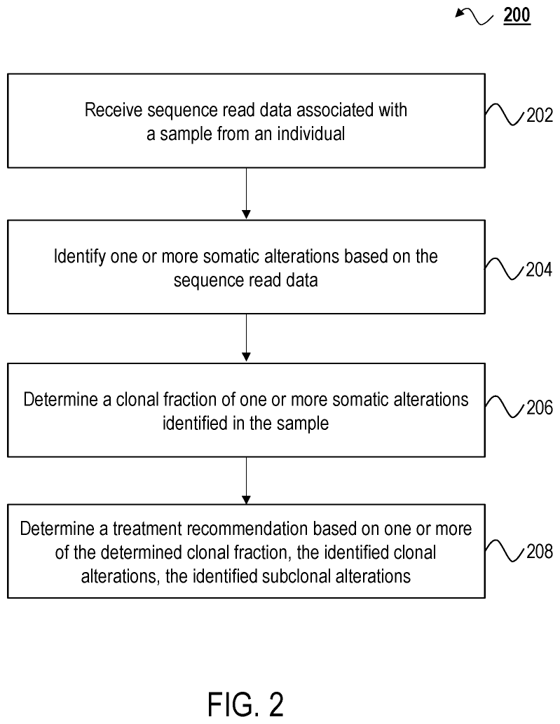

The clonal fraction and tumor fraction of somatic alterations in a sample may provide useful insight to healthcare providers and can be used to inform treatment decisions. FIG. 2 illustrates an exemplary process 200 flow for determining a treatment recommendation based on a clonal fraction or clonality of a somatic alteration in accordance with embodiments of this disclosure. As shown in the figure, the system can receive sequence read data associated with a sample from an individual at block 202. In one or more examples, the sample may be a solid biopsy sample or a liquid biopsy sample. In some instances, the sequence read data may be derived from single region sequencing (e.g., sequencing of a single tissue biopsy sample collected from the tumor of the individual). In some instances, the genomic data comprising sequence read data may be derived from multi-region sequencing (e.g., sequencing of multiple tissue biopsy samples collected from the tumor of the individual). In some instances, the genomic data comprising sequence read data may be derived from single cell sequencing data as opposed to bulk tumor sequencing. In some instances, the genomic data comprising sequence read data may be derived from sequencing circulating tumor DNA in a liquid biopsy sample. In some instances, the sequence read data may be derived from RNA in a liquid biopsy sample.

In some instances, the genomic data comprising sequence read data may be derived from targeted sequencing, e.g., targeted exome sequencing. In some instances, the genomic data comprising sequence read data may be derived from broad panel sequencing. In some instances, the sequence read data may be derived from whole genome or whole exome sequencing, e.g., as opposed to targeted exome sequencing or broad panel sequencing to increase the number of genomic features (e.g., the number of short variants, copy number alteration) detected. In one or more examples, the sequence read data may be received by the system as a BAM file.

At block 204 of FIG. 2, the system can identify one or more somatic alterations in the sample based on the received sequence read data. For instance, the sequence read data may be indicative of a presence or absence of one or more somatic alterations in a patient sample. In one or more examples, the sequence read data may also be indicative of the presence or absence of genomic events, such as copy number alterations, rearrangements, insertions, deletions, fusions, chromosomal aneuploidy, whole genome doubling, Catalogue Of Somatic Mutations In Cancer (COSMIC) mutational signatures, microsatellite instability (MSI) status, tumor mutational burden (TMB), or any combination thereof.

At block 206 of FIG. 2, the system can determine a clonal fraction of one or more somatic alterations identified in the sample. The clonal fraction can be determined in accordance with any of the methods described herein. In one or more embodiments, the system may further determine the clonality of the one or more somatic alterations. For instance, based on the clonal fraction determined at block 206, the system may determine whether the corresponding somatic alteration is clonal or subclonal. In one or more examples, the clonality may be determined as described above with respect process 100. The system can then determine which of the identified somatic alterations comprise clonal alterations and which comprise subclonal alterations.

At block 208 of FIG. 2, the system may determine a treatment recommendation based on one or more of the determined clonal fraction, the identified clonal alterations, and the identified subclonal alterations. Examples of treatment recommendations based on the clonal fraction and/or clonality of the somatic alterations are described in the following paragraphs.

In one or more examples, the system may determine that a sample from a patient includes an emerging subclonal alteration that is indicative of the patient developing a resistance to their current treatment. This type of emerging subclonal alteration may indicate that the patient is at risk of a clinical relapse in the near future. In this example, the system at Block 208, may determine that the treatment regimen for the patient should be modified to a therapy that targets the emerging subclonal alteration. For example, subclonal alterations in EGFR T790M or C797S, may indicate emerging resistance to EGFR inhibitor treatments. For instance, subclonal T790M alterations might indicate that the patient should be switched to Osimertinib or another next generation EGFR inhibitor. As another example, a subclonal alteration in KRAS, may result in a subset of the tumor responding to therapy. In this example, agents targeting a clonal phenomenon might be preferred (e.g., EGFR or ALK targeting therapies).

As another example, the system may determine that a sample from a patient includes an emerging subclonal alteration that is indicative of the patient developing a resistance to their current treatment. In this example, however, the emerging subclonal resistance alteration may not be associated with a target therapy. In such examples, regardless of whether there is an available targeted treatment for this therapy, the treatment regimen of the patient may be modified (e.g., at Block 208), as the subclonal alteration is indicative of the patient developing a resistance to the current treatment.

As another example, the system can determine which alterations correspond to subclonal alterations in a patient's sample. Identification of which alterations are subclonal might indicate which alterations are driving a patient's resistance to treatment. In one or more examples, the patient's current treatment regimen may be modified (e.g., at Block 208) based on the one or more identified subclonal alterations.

As another example, the system can determine which alterations correspond to subclonal alterations in a patient's sample. However, in some examples, the subclonal alterations may not be suitable for targeted therapy. For instance, because the subclonal alterations inherently represent a subset of tumor cells, there may not be a sufficient portion of tumor cells with the subclonal alteration associated with the targeted therapy for the therapy to be effective. In some instances, if an alteration is determined to be highly subclonal (e.g., the alteration is present in a very small fraction of tumor cells), the system may select therapies that target truncal/clonal alterations. This is because, the highly subclonal alteration may not be present in a sufficient number of tumor cells for the targeted therapy to be effective. In some instances, the system may consider the clonal fraction to determine a degree of clonality in order to inform the treatment decision. For instance, a threshold or range of thresholds may be set to indicate that whether an alteration in a sample is highly subclonal (e.g., present in a very small fraction of tumor cells) or subclonal (e.g., in a subset of tumor cells).

Aside from applying specific treatment recommendations, there are numerous avenues to apply information regarding clonality and the clonal fraction of somatic alterations in a sample. For instance, In one or more examples, process 100 and/or 200 can be applied to a plurality of samples taken from a patient over time. That is, the system may estimate the clonal fractions of the somatic alterations associated with a patient at multiple time points from samples associated with different bodily locations. This can provide longitudinal and spatial profiling of a clonal architecture of a patient. The clonal architecture may be used to track and understand tumor evolution and heterogeneity. Thus, the system may acquire data to track the changes of the disease over time, from early stages (tumor initiation) to metastasis/relapse to inform the patient's journey.

As another example, the system may determine that a sample from a patient includes one or more subclonal alterations. The presence of a subclonal alteration may be indicative of a poor prognosis. Additionally, the presence of the subclonal alteration(s) may be indicative of tumor heterogeneity or emerging resistance that signals poor progression-free survival (PFS) and overall survival (OS) outcomes. Treatment decisions may be made based on this prognostic information.

As another example, the system can determine which alterations correspond to subclonal alterations for a plurality of samples from a population of patients. Identification of subclonal alterations in a population of patients may be analyzed to determine resistance mechanisms (e.g., focusing on subclonal alterations) based on a samples in the absence of paired biopsies. The system may then generate a list of the identified resistance for drug discovery efforts to combat or treat resistance.

As another example, the system can determine which alterations correspond to clonal alterations and which alterations correspond to subclonal alterations in a patient's sample taken at the time when the patient is diagnosed (e.g., pre-treatment). Clonality of alterations in samples taken at diagnosis may inform strategies for treatment, including potential combination therapies. In some instances, clonal events may be prioritized for targeted therapy (where available). In some instances, clonality determinations may also be used to monitor the disease. In some instances, clonal determinations may be particularly useful for liquid biopsies. This is because liquid biopsies integrate the DNA from all tumor sites, thus, liquid biopsies may provide a comprehensive understanding of the overall fraction of tumor cells in the body that might respond to therapy.

For instance, in one or more examples, the system may determine that a sample from a patient includes one or more subclonal alterations. Based on the determined clonality of the somatic alterations, the system can predict a patient's sensitivity to treatment and accordingly how this sensitivity may impact the patient's survival outcomes. For instance, clonal KEAP1 in the presence of STK11 alterations may be associated with reduced sensitivity to treatment with immune checkpoint inhibitors. In some instances, a subclonal KEAP1 alteration in the presence of an STK11 alteration, may be predicted to have a similar survival outcome as a wild-type alteration and may be associated with a decreased overall survival.

### Methods for Distinguishing Somatic and Germline Alterations

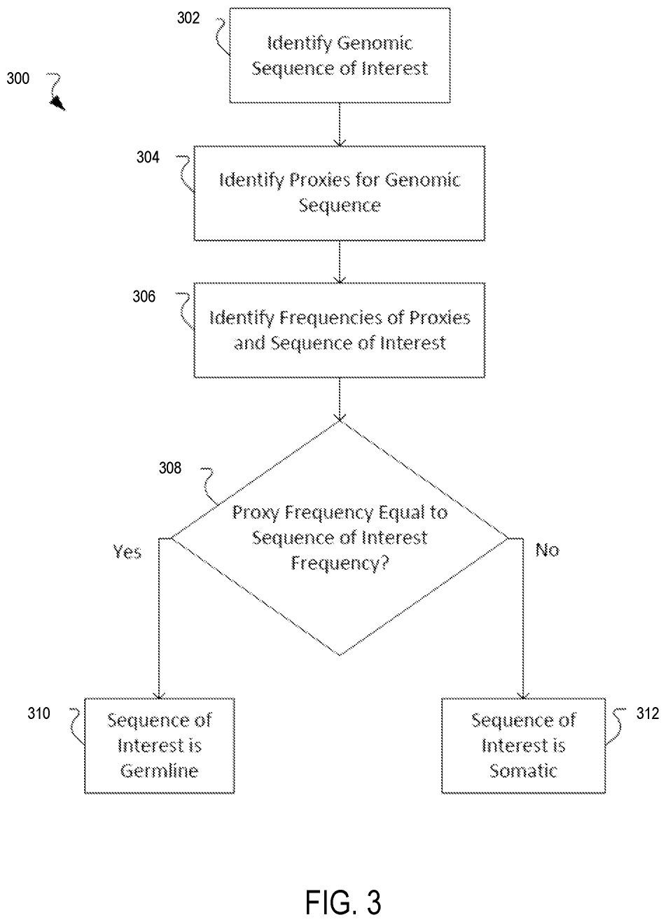

Embodiments of the present disclosure may use any suitable technique to determine whether a short variant identified in a sample is a somatic or germline alteration. FIG. 3 is a flowchart for an exemplary process 300 to distinguish germline and somatic genomic alterations.

Process 300 can be performed, for example, using one or more electronic devices implementing a software platform. In some examples, process 300 is performed using a client-server system, and the blocks of process 300 are divided up in any manner between the server and a client device. In other examples, the blocks of process 300 are divided up between the server and multiple client devices. Thus, while portions of process 300 are described herein as being performed by particular devices of a client-server system, it will be appreciated that process 300 is not so limited. In other examples, process 300 is performed using only a client device or only multiple client devices. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail below) are exemplary by nature and, as such, should not be viewed as limiting.

The process 300 begins with identifying a genomic region of interest (Block 302). In some implementations, Block 302 involves identifying a region of interest from within a larger genomic region.

Determining a genomic sequence from a physical sample can be accomplished in a variety of ways. One such way is described in U.S. Pat. No. 9,340,830, and another is described in U.S. Pat. Pub. 2017/0356053, the entireties of both of which are incorporated by reference herein. More generally, there is a category of machines that are operable to determine the genetic sequence of an input sample called genomic sequencers, including but not limited to sequencing platforms offered by ROCHE, SOLiD, PACBIO, ILLUMINA, and others. Moreover, there are a variety of known sub-regions of human and other organisms' genomes that are known to be relevant to a variety of medical conditions.

The techniques described herein do not depend on the use of a particular sequencing platform or particular sequencing techniques, and any of these machines and accompanying techniques may be used in Block 302.

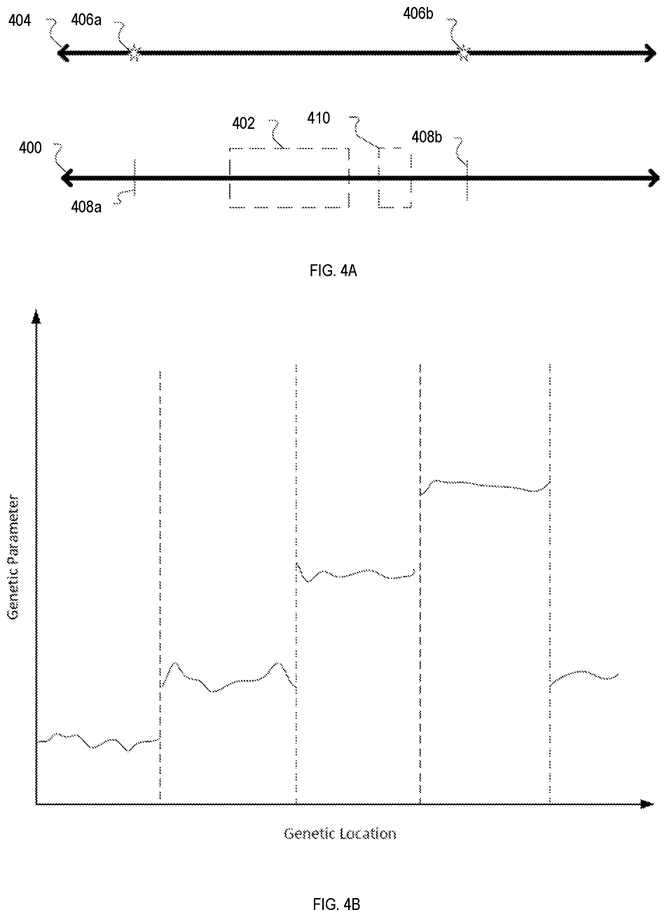

Referring briefly to FIG. 4A, in some implementations, a region of interest 402 is identified to correspond to a known genetic locus within a reference genome 404. In some implementations, the region of interest 402 corresponds to a mutation with respect to the reference sequence 404; i.e., a subsection of the genomic region 400 other than a polymorphic region that has a different genetic sequence from that of the corresponding part of reference sequence 404. In some implementations, the sequence of interest corresponds to a gene relevant to a medical condition that the patient possesses. In some implementations, the region of interest 402 is an oncogene or portion thereof. In some examples, the region of interest can include coding and non-coding regions (e.g., intronic, etc.).

In Block 304, one or more proxy genomic sequences for the genomic sequence are identified (Block 304). Referring to FIG. 4A, one characterization of a proxy 410 is a sequence at a genetic locus that is (a) physically close to the sequence of interest 402, and (b) known to encode germline genetic information. An alternative characterization is to require that the proxy 410 is known to encode somatic genetic information. For convenience, this document will assume that proxies 410 encode germline information unless otherwise specified, but those skilled in the art will appreciate the equivalence of the two approaches.

The germline status of a particular candidate proxy sequence may be known from research literature, publicly available databases (e.g., dbSNP (available at www.ncbi.nlm.nih.gov/snp/) or gnomAD (available at gnomad.broadinstitute.org)), or may be discovered by other ab initio means. On the other hand, somatic variants can be identified from matched tumor/normal samples; i.e., samples from the same patient that contain both tumor DNA and non-tumor (“normal”) DNA. In particular, variants seen in tumor DNA but not in corresponding normal DNA are necessarily somatic. Known somatic variants may also be discovered by other ab initio means.

Referring to FIG. 4B, in some implementations Block 304 is performed by employing a segmentation process. In such a process, the portion of the patient's genome is partitioned into segments (delineated by dashed lines in FIG. 4B) based on a genetic parameter. The segments are defined such that the parameter values in a particular segment are all equal (within a desired threshold). In some implementations, the genetic parameter used to segment the input includes copy number, frequency of an allele or sub-allelic segment of interest, or others.

A variety of segmentation procedures are known in the art. For example, iSeg (described in Girimurugan et al., iSeg: an Efficient Algorithm for Segmentation of Genomic and Epigenomic Data, BMC Bioinformatics 19:131 (2018), the entirety of which is incorporated herein), CBS (described in Olshen et al., Circular Binary Segmentation for the Analysis of Array-Based DNA Copy Number Data, Biostatistics 2004 October; 5(4):557-72, the entirety of which is incorporated by reference herein), and SLMSuite (described in Orlandini et al., SLMSuite: A Suite of Algorithms for Segmenting Genomic Profiles, BMC Bioinformatics 18:321 (2017), the entirety of which is incorporated by reference herein) are three among many such algorithms.

Referring back to FIG. 4A, in some implementations only proxies 410 that lie on the same segment as the region of interest 402 are identified. In some implementations the proxies 410 include all known germline SNPs lying on the same segment as the region of interest 402. In some implementations the proxies 410 include all known germline alleles on the same segment as the region of interest 402. In some implementations, only proxies 410 that are no more than a pre-determined number of bases away from the region of interest 402 are identified.

In Block 306, the frequencies of sequences from the region of interest 402 and the proxies 410 are identified. Here, “frequency” refers to normalized statistical frequency—for example, the number of occurrences of a sequence or proxy within the sample, divided by the total number of occurrences of any sequence at the same genomic locus. In some implementations, several frequency measurements may be made. When using several proxies, outlier proxy frequencies may be discarded and the remaining frequencies may be combined to a single statistical centrality measure (e.g., mean, median, mode, or others) so that Block 308 involves a single numerical comparison.

In decision 308, the proxy frequency or frequencies (for example, a centrality measure of the observed frequencies of the one or more proxy sequences) are compared to the frequency or frequencies of the region of interest to determine if they are equal. Here and throughout this application “equal” includes equal to within a desired threshold that can routinely be determined based on desired selectivity and specificity of the process 300. The threshold may be set, for example, using a statistical threshold or statistical test selected by one skilled in the art. If several proxies 110 are used and individual comparisons are made instead of combining the proxy frequencies as described above, then a decision 308 results in a “yes” if a certain proportion of the comparisons (e.g., greater than 50%, greater than 55%, greater than 60%, greater than 65%, greater than 70%, greater than 75%, greater than 80%, greater than 85%, greater than 90%, or greater than 95%) are equal.

If the proxy frequency is equal to the frequency of the sequence of interest, then the sequence of interest is classified as germline (Block 310). Otherwise, the sequence of interest is classified as somatic (Block 312). Alternatively, if proxies 410 were selected to be known to encode somatic information (instead of germline), then equal frequencies are interpreted as the sequence of interest being somatic and unequal frequencies is interpreted as the sequence of interest being germline.

In some implementations, the comparison in decision 308 may also be used to eliminate potentially erroneous classifications. In particular, the frequency of a true somatic variant is necessarily less than a true germline variant, because both tumor and non-tumor DNA contribute to a germline variant's frequency count, while only tumor DNA contributes to a somatic variant's frequency count. Thus, in some implementations, if the frequency of the sequence of interest is greater than the proxy frequency, then sequence of interest is classified as germline.

In some implementations, the comparison of Block 308 may be indirectly performed by way of a statistical model. For example, if the median allele frequency of a collection of proxies is used as the central measure of Block 306, then a logistic regression model may be constructed that describes the difference of the allele frequency of the sequence of interest to the median allele frequency of the proxies. In some implementations, this logistic regression model can be constructed from a collection of matched tumor/normal samples, such that the difference described in the previous sentence is proportional to

$$log ( \frac{p}{1 - p} ) ,$$

log where p represents the probability that the sequence of interest comprises a somatic variant.

The rationale underlying this characterization is that each proxy is physically close to the sequence of interest in the patient's genome. Thus, it is likely that the proxy and the sequence of interest experience the same or similar genomic dynamics or mutations, such as duplication events or deletions. Rather than attempting to model the specific dynamics of the sequence of interest to correlate observed frequencies with germline/somatic status, this approach replaces such a model with a direct empirical measurement.

FIG. 5 provides another non-limiting example of a process 500 for distinguishing somatic alterations from germline alterations. In one or more examples, distinguishing somatic alterations from germline alterations be based on a somatic-germline score. See U.S. Pat. No. 9,792,403, which is incorporated herein in its entirety.

Process 500 can be performed, for example, using one or more electronic devices implementing a software platform. In some examples, process 500 is performed using a client-server system, and the blocks of process 500 are divided up in any manner between the server and a client device. In other examples, the blocks of process 500 are divided up between the server and multiple client devices. Thus, while portions of process 500 are described herein as being performed by particular devices of a client-server system, it will be appreciated that process 500 is not so limited. In other examples, process 500 is performed using only a client device or only multiple client devices. In process 500, some blocks are, optionally, combined, the order of some blocks is, optionally, changed, and some blocks are, optionally, omitted. In some examples, additional steps may be performed in combination with the process 500.

Accordingly, the operations as illustrated (and described in greater detail below) are exemplary by nature and, as such, should not be viewed as limiting.

At Block 502 in FIG. 5, the system can determine a sequence coverage input (SCI), based on a number of reads of a selected genomic segment of one or more specified subgenomic intervals in the sample. SCI can refer to the number of mapped reads that cover a region of interest (e.g., 500× coverage for a region or base). In one or more examples, the system can assign SCI values to the selected genomic segments (e.g., a plurality of subgenomic intervals).

In one or more examples, system may be configured to capture data on a genomic sequence coverage for specified subgenomic intervals. The system can define a variable for the SCI based on the values for sequence coverage at the specified subgenomic intervals. In one or more examples, the system includes a user interface display configured to accept user input to define the specified subgenomic intervals. In other embodiments, the subgenomic intervals can be pre-defined as part of genetic testing and/or analysis. Further, the system can also be configured to identify the subgenomic intervals to analyze automatically (e.g., based on segmentation analysis, etc.). Once the subgenomic intervals are specified, the system can capture a value for sequence coverage for each of a plurality of specified subgenomic intervals. The captured values can be normalized, averaged, or weighted to prevent outlier values from skewing subsequent calculations.

In one or more examples, the system and/or system components are configured to fit the genome-wide copy number model to the SCI using Equation 5:

$$log {Ratio}_{i} = {log}_{2} \frac{{pC}_{i} + 2 ( 1 - p )}{p ψ + 2 ( 1 - p )} \qquad ( 5 )$$

where ψ is tumor ploidy, C is a copy number, and p is a sample purity. The system and/or system components can calculate ψ as =(ΣiliCi)/Σili, where li is the length of a genomic segment. As used herein, the tumor ploidy can refer to the average number of copies of each chromosome; the copy number can refer to the number of estimated copies for a genomic region; and sample purity can refer to the estimated tumor fraction for the sample (fraction of nuclei that are from tumor cells).

In one or more embodiments, the system may further determine a mutant allele copy value (me) and a reference copy value (wc). For instance, the system can use the allele fraction, estimated copy number, minor allele fraction, and for each possible estimate of me, we, and germline/somatic, determine a value that best corresponds to the data. For example, if a variant is somatic in a copy number 3 segment, the me could be 0, 1, 2, or 3 which would correspond to a we of 3, 2, 1, or 0, respectively. In such examples, mutant copies (me) can be based on the count of the number of tumor copies with the mutation (e.g., two of three tumor copies). Reference copy values (wc) would be the number of non-mutated copies (e.g., one of three).

At Block 504 in FIG. 5, the system can determine a single nucleotide polymorphism (SNP) allele frequency input (SAFI), based on a SNP allele frequency for each of a plurality of selected germline SNPs in the sample. In some embodiments, the SNP allele frequency corresponds to a portion of reads supporting a particular allele. For example, in the case of 500 reads supporting the A allele and 250 supporting the B allele, the B allele frequency would be 0.333. A SAFI may be the smaller of the A or B allele at a germline SNP (e.g., if the A allele corresponds to a value of 0.63 and B corresponds to a value of 0.37, then the minor allele frequency would be 0.37). Values near 0.5 indicate balanced copy numbers (e.g., 1 and 1 for the A allele and B allele or 2 and 2 for the A allele and B allele).

In one or more examples, the system can be configured to derive an allele frequency value according to specification of germline SNPs in the tumor sample. The system can define a variable for a SAFI based on the values for allele frequency for the selected germline SNPs. In some embodiments, the system specifies the germline SNPs on which to capture values for allele frequency (e.g., based on pre-specified selection, automatically based on analysis of the tumor sample, etc.). In other embodiments, the user interface can also be configured to accept selection of germline SNPs within genetic sequencing information obtained on, for example, a tumor sample.

The system can also be configured to fit the genome-wide copy number model to the SAFI using Equation 6:

$$AF = \frac{pM + 1 ( 1 - p )}{pC + 2 ( 1 - p )} \qquad ( 6 )$$

where AF is allele frequency, p is the sample purity, and M is the minor allele frequency. Various fitting methodologies can be executed by the system to determine a g value indicative of a germline or somatic origin of the sample (e.g., Markov chain Monte Carlo (MCMC) algorithm, e.g., ASCAT (Allele-Specific Copy Number Analysis of Tumors), OncoSNP, or PICNIC (Predicting Integral Copy Numbers In Cancer).

At Block 506 in FIG. 5, the system can determine a variant allele frequency input (VAFI), based on an allele frequency for an alteration identified in the sample.

For example, the system can be configured to capture and/or calculate additional values from genetic sequence information (including, e.g., captured from testing systems and/or components or generated by the characterization system directly). In one or more examples, the system can capture the VAFI for a given variant (e.g., a mutation) from testing data. In another example, the system can generate the data for capturing the allele frequency responsive to genetic sequence testing performed on the sample.

At Block 508 in FIG. 5, the system can obtain values based on the SCI, SAFI, and the VAFI for each of: a genomic segment total copy number (C value) for a plurality of genomic segments; a genomic segment minor allele copy number (M value) for a plurality of genomic segments in the sample; and a sample purity (p). In some embodiments the system may determine the C value, M value and/or P value. In some embodiments, the system may obtain the C value, M value, and/or p value.

At Block 510 in FIG. 5, the system can determine a tumor type (e.g., somatic, germline, and not-distinguishable) based on the genetic sequencing data. In some embodiments this is achieved without resort to physical analysis of a control sample to determine, e.g., purity. For example, the system can calculate a value for a mutation type (“g”—e.g., a value that is indicative of a variant being somatic, germline, or not-distinguishable) by executing a function on the acquired and/or calculated values for VAFI, p, C, and M. Based on the output value of g, the system can classify the mutation type. In one example, a g value equal or approximately equal to 0 is classified by the system as somatic variant. In another example, a g value equal or approximately equal to 1 is classified by the system as a germline variant. Values of g between 0 and 1 (e.g., 0.4-0.6) can be classified by the system as not-determinable.

In some implementations, the system can also be configured to determine a confidence level associated with any calculation and/or calculated value (e.g., based on statistical analysis of the input(s) and computational values used to derive an output). The system can use determinations on the confidence of calculations and/or calculated values in interpreting classification outputs. In one example, the not-determinable range of values can be increased where the degree of confidence associated with the calculation of the g value is low. In another example, the not-determinable range of values can be decreased where the degree of confidence associated with the calculation of the g value is high.

### Additional Methods for Determining a Clonal Fraction of a Somatic Alteration

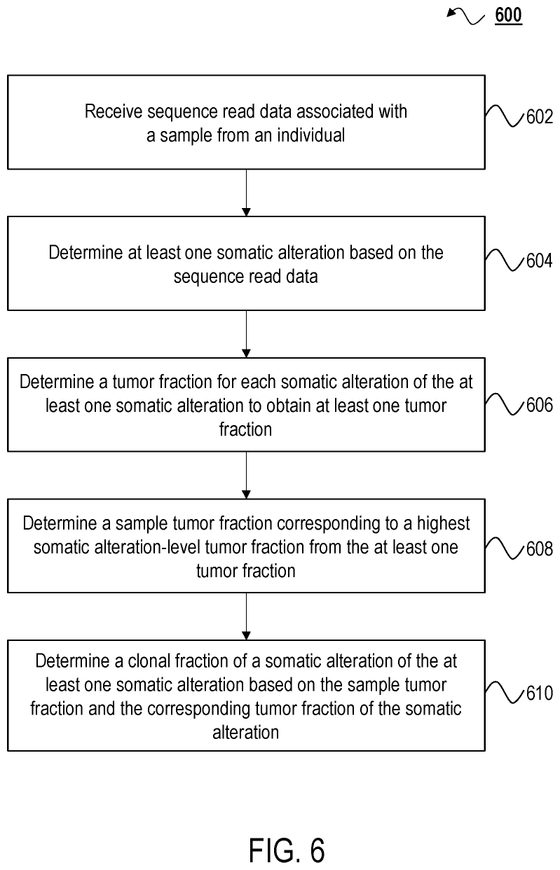

FIG. 6 provides a non-limiting example of a process 600 for determining a clonal fraction of one or more somatic alterations in a sample, in accordance with embodiments of the present disclosure. Process 600 can be used to determine a clonal fraction of one or more somatic alterations in a sample taken from an individual. In some examples, the process 600 can be used to determine a clonality of one or more somatic alterations.

Process 600 can be performed, for example, using one or more electronic devices implementing a software platform. In some examples, process 600 is performed using a client-server system, and the blocks of process 600 are divided up in any manner between the server and a client device. In other examples, the blocks of process 600 are divided up between the server and multiple client devices. Thus, while portions of process 600 are described herein as being performed by particular devices of a client-server system, it will be appreciated that process 600 is not so limited. In other examples, process 600 is performed using only a client device or only multiple client devices. In process 600, some blocks are, optionally, combined, the order of some blocks is, optionally, changed, and some blocks are, optionally, omitted. In some examples, additional steps may be performed in combination with the process 600. Accordingly, the operations as illustrated (and described in greater detail below) are exemplary by nature and, as such, should not be viewed as limiting.

At Block 602 of FIG. 6, the system can receive sequence read data associated with a sample from an individual. In one or more examples, the sample may be a solid biopsy sample or a liquid biopsy sample. In some instances, the sequence read data may be derived from single region sequencing (e.g., sequencing of a single tissue biopsy sample collected from the tumor of the individual). In some instances, the genomic data comprising sequence read data may be derived from multi-region sequencing (e.g., sequencing of multiple tissue biopsy samples collected from the tumor of the individual). In some instances, the genomic data comprising sequence read data may be derived from single cell sequencing data as opposed to bulk tumor sequencing. In some instances, the genomic data comprising sequence read data may be derived from sequencing circulating tumor DNA in a liquid biopsy sample. In some instances, the sequence read data may be derived from RNA in a liquid biopsy sample.

In some instances, the genomic data comprising sequence read data may be derived from targeted sequencing, e.g., targeted exome sequencing. In some instances, the genomic data comprising sequence read data may be derived from broad panel sequencing. In some instances, the sequence read data may be derived from whole genome or whole exome sequencing, e.g., as opposed to targeted exome sequencing or broad panel sequencing to increase the number of genomic features (e.g., the number of short variants, copy number alteration) detected. In one or more examples, the sequence read data may be received by the system as a BAM file.

In one or more examples, the sequence read data may be indicative of a presence or absence of one or more somatic alterations in a patient sample. In one or more examples, the sequence read data may also be indicative of the presence or absence of genomic events, such as copy number alterations, rearrangements, insertions, deletions, fusions, chromosomal aneuploidy, whole genome doubling, Catalogue Of Somatic Mutations In Cancer (COSMIC) mutational signatures, microsatellite instability (MSI) status, tumor mutational burden (TMB), or any combination thereof.

In some examples, the system may further perform a quality control process. During the quality control process, the system determines whether the sample meets minimum requirements associated with reliability for processing the sample. In one or more examples, if the sample fails to meet one or more of quality control metrics, the system may not proceed to determine the clonal fraction for the sample. In some examples, the quality control process may correspond to quality control process described with respect to block 102 of process 100.

At Block 604 of FIG. 6, the system can determine at least one somatic alteration based on the sequence read data. For instance, the sample may comprise somatic alterations and the sequence read data may be used to identity the somatic alterations present in the sample. In some instances, the determination of the somatic alteration may be based on a tumor type determination as described above with respect to process 100 (e.g., Blocks 104-106), process 300, and process 500. Due to the volume of data associated with the sequence read data and the computational complexity associated with analyzing the sequence read data, the system may perform this determination using one or more electronic devices.

At block 606 of FIG. 6, the system determines a tumor fraction for each somatic alteration of the at least one somatic alteration to obtain at least one tumor fraction. The tumor fraction (VTF) for a specific somatic alteration may be based on the allele frequency, the mutant allele copy value, and the reference copy value of the somatic alteration. In one or more examples, the system can use Equation 2, provided above, along with the me value, we value, and AF determined by the system (e.g., at Block 104 of process 100 or during process 500) to estimate or determine the tumor fraction for a somatic alteration identified in the sample. The tumor fraction can be estimated for one or more of the at least one somatic alterations determined at Block 604. In some embodiments, the tumor fraction can be estimated for each of the somatic alterations identified in Block 604. In one or more examples, block 606 may correspond to the description provided above with respect to block 110 of process 100.

At block 608 of FIG. 6, the system determines a sample tumor fraction corresponding to a highest tumor fraction from the at least one tumor fraction. The sample tumor fraction may be indicative of the fraction of cells that corresponds to tumor cells in the sample. In one or more embodiments, the highest tumor fraction (TFₘₐₓ) from the tumor fractions VTFᵢ (e.g., tumor fractions determined in Block 110) for the somatic alterations identified in a sample, is set to the tumor fraction (TF) estimate for the sample according to Equation 3 provided above. In one or more examples, block 608 may correspond to the description provided above with respect to block 112 of process 100.

As discussed above, determining a sample tumor fraction may be challenging, particularly for data derived from targeted, panel-based NGS. This is because, unlike whole genome sequencing (WGS) or whole exome sequencing (WES), which sequence the entire genome, panel-based NGS selects segments of the genome for analysis. As a result, the data derived from panel-based NGS and/or lower depth coverage may not be as comprehensive as data derived from WES and WGS. In instances where the clonal fraction and clonality are determined based on sequencing data obtained from panel-based NGS, embodiments of the present disclosure rely on the maximum tumor fraction (VTFₘₐₓ) of the identified somatic variants as a proxy for the sample tumor fraction.

As discussed above, early mutation events or initiating driver events (e.g., such as an EGFR-L858R) are expected to be present in each of the tumor cells. In some embodiments, early events or initiating driver events may be expected to be present in a majority of the tumor cells. Thus, the highest tumor fraction selected from the tumor fractions for each of the somatic alterations identified in a sample can be indicative of an initiating driver event or an early mutation event, e.g., because the highest tumor fraction is the alteration present in the greatest portion of the somatic tumor cells. Thus, the highest tumor fraction may be a reliable approximation for the tumor fraction because each of the tumor cells are expected to contain the corresponding alteration. In this manner, embodiments of the present disclosure can provide an accurate estimate of a sample tumor fraction in the absence of data obtained via WGS or WES techniques.

At block 610 of FIG. 6, the system determines a clonal fraction of a somatic alteration of the at least one somatic alteration based on the sample tumor fraction and the corresponding tumor fraction of the somatic alteration. For example, a clonal fraction may be determined based on Equation 4, provided above. In one or more examples, block 610 may correspond to the description provided above with respect to block 114 of process 100.

In one or more embodiments, the system may further determine a clonality of one or more of the somatic alterations based on the corresponding clonal fraction. For example, the somatic alteration may be determined to be clonal if the corresponding clonal fraction is greater than or equal to a threshold. In some instances, the threshold may be approximately 0.5. The sample may be subclonal if the clonal fraction is less than the threshold. In some instances, the threshold may be a different threshold, or may be a range of thresholds. In one or more examples, the clonality can be determined as described above with respect to block 116.

Accordingly, embodiments of the present disclosure provide systems and methods for determining a clonal fraction of one or more somatic alterations present in a sample. As discussed, the clonal fraction and/or clonality of the one or more somatic alterations may be used to determine and/or modify treatment options for patients. For instance, the clonal fraction and/or clonality determined using process 600 may be used to determine treatment for a patient as described above. Moreover, embodiments of the present disclosure may provide methods for accurately determining the clonal fraction and/or clonality of one or more somatic alterations of a sample based on targeted, panel-based NGS. As discussed above, determining an accurate sample tumor fraction based on panel-based NGS overcomes the challenges associated with determining an accurate clonal fraction and/or clonality of a sample based on data derived from panel-based NGS.

In some instances, the disclosed methods may be used to identify variants in the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (viii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for determining a clonal fraction may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for determining a clonal fraction may be used to predict genetic disorders in fetal DNA. (e.g., for invasive or non-invasive prenatal testing). For example, sequence read data obtained by sequencing fetal DNA extracted from samples obtained using invasive amniocentesis, chorionic villus sampling (cVS), or fetal umbilical cord sampling techniques, or obtained using non-invasive sampling of cell-free DNA (cfDNA) samples (which comprises a mix of maternal cfDNA and fetal cfDNA), may be processed according to the disclosed methods to identify variants, e.g., copy number alterations, associated with, e.g., Down Syndrome (trisomy 21), trisomy 18, trisomy 13, and extra or missing copies of the X and Y chromosomes.

In some instances, the disclosed methods for determining a clonal fraction may be used to select a subject (e.g., a patient) for a clinical trial based on the clonal fraction determined for one or more gene loci. In some instances, patient selection for clinical trials based on, e.g., determination of a clonal fraction associated with one or more gene loci, may accelerate the development of targeted therapies and improve the healthcare outcomes for treatment decisions.

In some instances, the disclosed methods for determining a clonal fraction may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the targeted therapy (or anti-cancer target therapy) may comprise abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the disclosed methods for determining a clonal fraction may be used in treating a disease (e.g., a cancer) in a subject. For example, in response to determining the clonal fraction using any of the methods disclosed herein, an effective amount of an anti-cancer therapy or anti-cancer treatment may be administered to the subject.

In some instances, the disclosed methods for determining a clonal fraction may be used for monitoring disease progression or recurrence (e.g., cancer or tumor progression or recurrence) in a subject. For example, in some instances, the methods may be used to determine a clonal fraction in a first sample obtained from the subject at a first time point, and used to determine a clonal fraction in a second sample obtained from the subject at a second time point, where comparison of the first determination of the clonal fraction and the second determination of the clonal fraction allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the clonal fraction.

In some instances, the value of the clonal fraction determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for determining a clonal fraction of a somatic alteration in a sample may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for determining a clonal fraction as part of a genomic profiling process (or inclusion of the output from the disclosed methods for determining a clonal fraction as part of the genomic profile of the subject) can improve the validity of, e.g., disease detection calls and treatment decisions, made on the basis of the genomic profile by, for example, independently determining the clonal fractions of one or more somatic alterations in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions—deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27(11):1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequencing—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Systems

Also disclosed herein are systems designed to implement any of the disclosed methods for determining a clonal fraction of one or more somatic alterations in a sample from a subject. The systems may comprise, e.g., one or more processors, and a memory unit communicatively coupled to the one or more processors and configured to store instructions that, when executed by the one or more processors, cause the system to perform a process comprising: receiving, using one or more processors, sequence read data associated with the sample from the subject; determining, using the one or more processors, at least one somatic alteration based on the sequence read data; determining, using the one or more processors, a tumor fraction for each somatic alteration of the at least one somatic alteration based on the sequence read data to obtain at least one tumor fraction; determining, using the one or more processors, a sample tumor fraction, the sample tumor fraction corresponding to a highest tumor fraction from the at least one tumor fraction; determining, using the one or more processors, a clonal fraction of a somatic alteration of the at least one somatic alteration based on (1) the sample tumor fraction and (2) the corresponding tumor fraction of the somatic alteration.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLiD) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for determining a clonal fraction in any of a variety of samples as described herein (e.g., a tissue sample, biopsy sample, hematological sample, or liquid biopsy sample derived from the subject).

In some instances, the plurality of gene loci for which sequencing data is processed to determine the clonal fraction may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of the clonal fraction is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

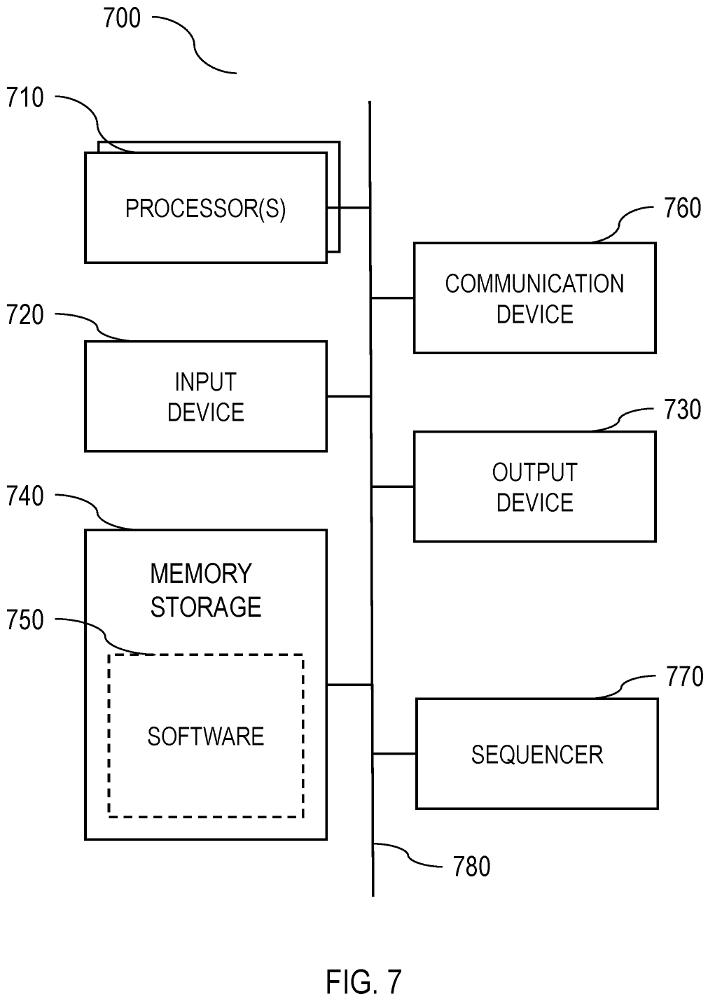

FIG. 7 illustrates an example of a computing device or system in accordance with one embodiment. Device 700 can be a host computer connected to a network. Device 700 can be a client computer or a server. As shown in FIG. 7, device 700 can be any suitable type of microprocessor-based device, such as a personal computer, workstation, server or handheld computing device (portable electronic device) such as a phone or tablet. The device can include, for example, one or more processor(s) 710, input devices 720, output devices 730, memory or storage devices 740, communication devices 760, and nucleic acid sequencers 770. Software 750 residing in memory or storage device 740 may comprise, e.g., an operating system as well as software for executing the methods described herein. Input device 720 and output device 730 can generally correspond to those described herein, and can either be connectable or integrated with the computer.

Input device 720 can be any suitable device that provides input, such as a touch screen, keyboard or keypad, mouse, or voice-recognition device. Output device 730 can be any suitable device that provides output, such as a touch screen, haptics device, or speaker.

Storage 740 can be any suitable device that provides storage (e.g., an electrical, magnetic or optical memory including a RAM (volatile and non-volatile), cache, hard drive, or removable storage disk). Communication device 760 can include any suitable device capable of transmitting and receiving signals over a network, such as a network interface chip or device. The components of the computer can be connected in any suitable manner, such as via a wired media (e.g., a physical system bus 780, Ethernet connection, or any other wire transfer technology) or wirelessly (e.g., Bluetooth®, Wi-Fi®, or any other wireless technology).

Software module 750, which can be stored as executable instructions in storage 740 and executed by processor(s) 710, can include, for example, an operating system and/or the processes that embody the functionality of the methods of the present disclosure (e.g., as embodied in the devices as described herein).

Software module 750 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 740, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 750 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

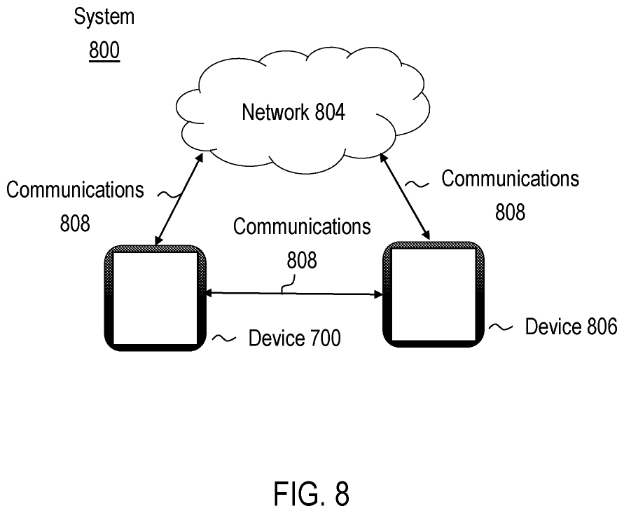

Device 700 may be connected to a network (e.g., network 804, as shown in FIG. 8 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 700 can be implemented using any operating system, e.g., an operating system suitable for operating on the network. Software module 750 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 710.

Device 700 can further include a sequencer 770, which can be any suitable nucleic acid sequencing instrument.

FIG. 8 illustrates an example of a computing system in accordance with one embodiment. In system 800, device 700 (e.g., as described above and illustrated in FIG. 7) is connected to network 804, which is also connected to device 806. In some embodiments, device 806 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio® RS system.

Devices 700 and 806 may communicate, e.g., using suitable communication interfaces via network 804, such as a Local Area Network (LAN), Virtual Private Network (VPN), or the Internet. In some embodiments, network 804 can be, for example, the Internet, an intranet, a virtual private network, a cloud network, a wired network, or a wireless network. Devices 700 and 806 may communicate, in part or in whole, via wireless or hardwired communications, such as Ethernet, IEEE 802.11b wireless, or the like. Additionally, devices 700 and 806 may communicate, e.g., using suitable communication interfaces, via a second network, such as a mobile/cellular network. Communication between devices 700 and 806 may further include or communicate with various servers such as a mail server, mobile server, media server, telephone server, and the like. In some embodiments, Devices 700 and 806 can communicate directly (instead of, or in addition to, communicating via network 804), e.g., via wireless or hardwired communications, such as Ethernet, IEEE 802.11b wireless, or the like. In some embodiments, devices 700 and 806 communicate via communications 808, which can be a direct connection or can occur via a network (e.g., network 804).

One or all of devices 700 and 806 generally include logic (e.g., http web server logic) or are programmed to format data, accessed from local or remote databases or other sources of data and content, for providing and/or receiving information via network 804 according to various examples described herein.

### EXEMPLARY IMPLEMENTATIONS

Exemplary implementations of the methods and systems described herein include:
1. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads;
   determining, using the one or more processors, at least one somatic alteration based on the sequence read data;
   determining, using the one or more processors, a tumor fraction for each somatic alteration from the at least one somatic alteration, such that at least one tumor fraction is obtained;
   identifying, using the one or more processors, a tumor fraction with a highest value from the at least one tumor fraction as a sample tumor fraction; and
   determining, using the one or more processors, a clonal fraction for each somatic alteration from the at least one somatic alteration based on (1) the sample tumor fraction, and (2) a corresponding tumor fraction for each somatic alteration.
2. The method of clause 1, wherein the corresponding tumor fraction is associated with a corresponding somatic alteration of the plurality of somatic alterations, the corresponding tumor fraction determined based on an allelic frequency of the corresponding somatic alteration, a mutant copy value of the corresponding somatic alteration, and a wildtype copy value of the corresponding somatic alteration.
3. The method of clause 2, wherein one or more of the allelic frequency, the mutant copy value, or the wildtype copy value is based on the sequence read data.
4. The method of any of clauses 1 to 3, further comprising classifying, using the one or more processors, the corresponding somatic alteration of the sample as clonal if a corresponding clonal fraction is greater than a threshold.
5. The method of any of clauses 1 to 4, further comprising classifying, using the one or more processors, the corresponding somatic alteration of the sample as subclonal if a corresponding clonal fraction is less than a threshold.
6. The method of any of clause 4 or clause 5, wherein the threshold corresponds to a value of 0.5.
7. The method of any of clauses 1 to 6, further comprising determining whether a quality control (QC) metric of the sample exceeds a quality control threshold.
8. The method of clause 7, wherein the QC metric is associated with one or more of: a sample purity; a sample noise; a sample aneuploidy; single-nucleotide polymorphism (SNP) data; an absence of somatic alterations; and an inconclusive copy number estimate.
9. The method of any one of clauses 1 to 8, wherein the subject is suspected of having or is determined to have cancer.
10. The method of clause 9, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
11. The method of clause 9, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.
12. The method of clause 9, further comprising treating the subject with an anti-cancer therapy.
13. The method of clause 12, wherein the anti-cancer therapy comprises a targeted anti-cancer therapy.
14. The method of clause 13, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.
15. The method of any one of clauses 1 to 14, further comprising obtaining the sample from the subject.
16. The method of any one of clauses 1 to 15, wherein the sample comprises a tissue biopsy sample, a liquid biopsy sample, or a normal control sample.
17. The method of clause 16, wherein the sample is a liquid biopsy sample and comprises blood, plasma, cerebrospinal fluid, sputum, stool, urine, or saliva.
18. The method of clause 16, wherein the sample is a liquid biopsy sample and comprises circulating tumor cells (CTCs).
19. The method of any one of clauses 1 to 18, wherein the plurality of nucleic acid molecules comprises a mixture of tumor nucleic acid molecules and non-tumor nucleic acid molecules.
20. The method of clause 16, wherein the sample is a liquid biopsy sample and comprises cell-free DNA (cfDNA), circulating tumor DNA (ctDNA), or any combination thereof.
21. The method of clause 20, wherein the tumor nucleic acid molecules are derived from a tumor portion of a heterogeneous tissue biopsy sample, and the non-tumor nucleic acid molecules are derived from a normal portion of the heterogeneous tissue biopsy sample.
22. The method of clause 20, wherein the sample comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.
23. The method of any one of clauses 1 to 22, wherein the one or more adapters comprise amplification primers, flow cell adaptor sequences, substrate adapter sequences, or sample index sequences.
24. The method of any one of clauses 1 to 23, wherein the captured nucleic acid molecules are captured from the amplified nucleic acid molecules by hybridization to one or more bait molecules.
25. The method of clause 24, wherein the one or more bait molecules comprise one or more nucleic acid molecules, each comprising a region that is complementary to a region of a captured nucleic acid molecule.
26. The method of any one of clauses 1 to 25, wherein amplifying the one or more ligated nucleic acid molecules comprises performing a polymerase chain reaction (PCR) amplification technique, a non-PCR amplification technique, or an isothermal amplification technique.
27. The method of any one of clauses 1 to 26, wherein the sequencing comprises use of a massively parallel sequencing (MPS) technique, whole genome sequencing (WGS), whole exome sequencing, targeted sequencing, direct sequencing, or Sanger sequencing technique.
28. The method of clause 27, wherein the sequencing comprises massively parallel sequencing, and the massively parallel sequencing technique comprises next generation sequencing (NGS).
29. The method of any one of clauses 1 to 28, wherein the sequencer comprises a next generation sequencer.
30. The method of any one of clauses 1 to 29, wherein one or more of the plurality of sequencing reads overlap one or more gene loci within one or more subgenomic intervals in the sample.
31. The method of clause 30, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.
32. The method of clause 30 or clause 31, wherein the one or more gene loci comprise ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof.
33. The method of clause 30 or clause 31, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.
34. The method of any one of clauses 1 to 33, further comprising generating, by the one or more processors, a report indicating at least one clonal fraction of the clonal fraction for each somatic alteration from the at least one somatic alteration.
35. The method of clause 34, further comprising transmitting the report to a healthcare provider.
36. The method of clause 35, wherein the report is transmitted via a computer network or a peer-to-peer connection.
37. A method for determining a clonal fraction associated with a sample from a subject, the method comprising:
   receiving, using one or more processors, sequence read data associated with the sample from the subject;
   determining, using the one or more processors, at least one somatic alteration based on the sequence read data;
   determining, using the one or more processors, a tumor fraction for each somatic alteration of the at least one somatic alteration based on the sequence read data, such that at least one tumor fraction is obtained;
   determining, using the one or more processors, a sample tumor fraction, the sample tumor fraction corresponding to a highest tumor fraction from the at least one tumor fraction; and
   determining, using the one or more processors, a clonal fraction of a somatic alteration of the at least one somatic alteration based on (1) the sample tumor fraction and (2) a corresponding tumor fraction of the somatic alteration.
38. The method of clause 37, wherein the corresponding tumor fraction of the somatic alteration is determined based on an allelic frequency of the somatic alteration, a mutant copy value of the somatic alteration, and a wildtype copy value of the somatic alteration.
39. The method of clause 38, wherein one or more of the allelic frequency, the mutant copy value, or the wildtype copy value are obtained based on the sequence read data.
40. The method of any one of clauses 37 to 39, further comprising classifying, using the one or more processors, the somatic alteration as clonal if the clonal fraction is greater than a threshold.
41. The method of any one of clauses 37 to 40, further comprising classifying, using the one or more processors, the somatic alteration as subclonal if the clonal fraction is less than the threshold.
42. The method of any of clause 40 or clause 41, wherein the threshold corresponds to a value of 0.5.
43. The method of any of clauses 37 to 42, further comprising determining whether a quality metric (QC) of the sample exceeds a threshold.
44. The method of clause 43, wherein the QC metric is associated with one or more of: a sample purity; a sample noise; a sample aneuploidy; single-nucleotide polymorphism (SNP) data; an absence of somatic alterations; and an inconclusive copy number estimate.
45. The method of any of clauses 37 to 44, further comprising in accordance with a determination that the clonal fraction is associated with a subclonal alteration, assigning, using the one or more processors, a therapy for the subject based on the clonal fraction.
46. The method any of clauses 37 to 45, further comprising in accordance with a determination that the clonal fraction is associated with a subclonal alteration, administering, using the one or more processors, a treatment to the subject based on the clonal fraction.
47. The method of any of clause 45 or clause 46, wherein the therapy comprises a targeted therapy.
48. The method of any of clauses 37 to 44, further comprising in accordance with a determination that the clonal fraction is associated with a clonal alteration, assigning, using the one or more processors, a treatment to the subject based on the clonal fraction, wherein the therapy comprises a therapy configured to target the clonal alteration.
49. The method any of clauses 37 to 48, further comprising determining, using the one or more processors, a prognosis of the subject based on the clonal fraction.
50. The method any of clauses 37 to 49, further comprising monitoring, using the one or more processors, a progression of a disease of the subject based on the clonal fraction.
51. The method of clause 50, wherein the clonal fraction corresponding to a subclonal alteration is indicative of at least one of a poor prognosis, disease progression, and treatment resistance.
52. The method of any of clauses 37 to 51, further comprising in accordance with a determination that the clonal fraction is associated with a clonal alteration, identifying the respective alteration as a driver of disease in the subject.
53. The method any of clauses 37 to 52, further comprising predicting, using the one or more processors, one or more clinical outcomes based on the clonal fraction.
54. The method any of clauses 37 to 53, wherein the sequence read data for the subject is based on one or more of a broad panel sequencing panel, a targeted-exome sequencing panel, or a whole exome sequencing technique.
55. The method any of clauses 37 to 54, further comprising in accordance with a determination that the clonal fraction is associated with a subclonal alteration, recommending chemotherapy as a treatment.
56. The method any of clauses 37 to 55, wherein the sequence read data for the subject is derived from multiple biopsy samples or a single biopsy sample.
57. The method any of clauses 37 to 56, wherein the sequence read data for the subject is derived from single cell sequencing.
58. The method any of clauses 37 to 57, wherein the sequence read data for the subject is derived from circulating tumor DNA in a liquid biopsy sample.
59. The method of any of clauses 37 to 58, wherein the determination of the clonal fraction is used to diagnose or confirm a diagnosis of disease in the subject.
60. The method of any of clauses 37 to 59, wherein the disease is cancer.
61. The method of any of clauses 37 to 60, wherein the cancer is at least one of is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.
62. The method of any of clauses 37 to 61, further comprising selecting an anti-cancer therapy to administer to the subject based on the determination of the clonal fraction.
63. The method of any of clauses 37 to 62, further comprising determining an effective amount of an anti-cancer therapy to administer to the subject based on the determination of the clonal fraction.
64. The method of any of clauses 37 to 63, further comprising administering the anti-cancer therapy to the subject based on the determination of the clonal fraction.
65. The method of clause 64, wherein the anti-cancer therapy comprises chemotherapy, radiation therapy, immunotherapy, a targeted therapy, or surgery.
66. A method for diagnosing a disease, the method comprising: diagnosing that a subject has the disease based on a determination of the clonal fraction for a sample from the subject, wherein the clonal fraction is determined according to the method of any one of clauses 1 to 65.
67. A method of selecting an anti-cancer therapy, the method comprising: responsive to determining the clonal fraction for a sample from a subject, selecting an anti-cancer therapy for the subject, wherein the clonal fraction is determined according to the method of any one of clauses 1 to 65.
68. A method of treating a cancer in a subject, comprising: responsive to determining the clonal fraction associated with a sample from the subject, administering an effective amount of an anti-cancer therapy to the subject, wherein the clonal fraction is determined according to the method of any one of clauses 1 to 65.
69. A method for monitoring cancer progression or recurrence in a subject, the method comprising:
   determining a first clonal fraction in a first sample obtained from the subject at a first time point according to the method of any one of clauses 1 to 65;
   determining a second clonal fraction in a second sample obtained from the subject at a second time point; and comparing the first clonal fraction to the second clonal fraction, thereby monitoring the cancer progression or recurrence.
70. The method of clause 69, wherein the second the clonal fraction for the second sample is determined according to the method of any one of clauses 1 to 65.
71. The method of clause 69 or clause 70, further comprising selecting an anti-cancer therapy for the subject in response to the cancer progression.
72. The method of clause 69 or clause 70, further comprising administering an anti-cancer therapy to the subject in response to the cancer progression.
73. The method of clause 69 or clause 70, further comprising adjusting an anti-cancer therapy for the subject in response to the cancer progression.
74. The method of any one of clauses 61 to 73, further comprising adjusting a dosage of the anti-cancer therapy or selecting a different anti-cancer therapy in response to the cancer progression.
75. The method of clause 74, further comprising administering the adjusted anti-cancer therapy to the subject.
76. The method of any one of clauses 69 to 75, wherein the first time point is before the subject has been administered an anti-cancer therapy, and wherein the second time point is after the subject has been administered the anti-cancer therapy.
77. The method of any one of clauses 69 to 76, wherein the subject has a cancer, is at risk of having a cancer, is being routine tested for cancer, or is suspected of having a cancer.
78. The method of any one of clauses 69 to 77, wherein the cancer is a solid tumor.
79. The method of any one of clauses 69 to 78, wherein the cancer is a hematological cancer.
80. The method of any one of clauses 69 to 79, wherein the anti-cancer therapy comprises chemotherapy, radiation therapy, immunotherapy, a targeted therapy, or surgery.
81. The method of any one of clauses 1 to 65, further comprising determining, identifying, or applying the value of the clonal fraction associated with the sample as a diagnostic value associated with the sample.
82. The method of any one of clauses 1 to 65, further comprising generating a genomic profile for the subject based on the determination of the clonal fraction.
83. The method of clause 82, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.
84. The method of clause 82 or clause 83, wherein the genomic profile for the subject further comprises results from a nucleic acid sequencing-based test.
85. The method of any one of clauses 82 to 84, further comprising selecting an anti-cancer therapy, administering an anti-cancer therapy, or applying an anti-cancer therapy to the subject based on the generated genomic profile.
86. The method of any one of clauses 1 to 65, wherein the determination of the clonal fraction associated with the sample is used in making suggested treatment decisions for the subject.
87. The method of any one of clauses 1 to 65, wherein the determination of the clonal fraction associated with the sample is used in applying or administering a treatment to the subject.
88. A method comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, sequence read data for the plurality of sequence reads;
   determining, using the one or more processors, a quality control metric of the sample;
   determining, using the one or more processors, a plurality of tumor fraction estimates for each of a plurality of somatic alterations identified in the sample based on the sequence read data;
   determining, using the one or more processors, a sample tumor fraction, the sample tumor fraction corresponding to a highest tumor fraction estimate from the plurality of tumor fraction estimates;
   determining, using the one or more processors, a plurality of clonal fractions each clonal fraction corresponding to each of the plurality of somatic alterations; and
   classifying, using the one or more processors, the sample as clonal with respect to a particular somatic alteration of the plurality of somatic alterations if a corresponding clonal fraction is greater than a threshold.
89. A method for determining a clonality of alterations in a sample, the method comprising:
   receiving, at one or more processors, sequence read data for a plurality of sequence reads derived from the sample;
   determining, using the one or more processors, a plurality of tumor fractions for each of a plurality of somatic alterations based on the sequence read data;
   determining, using the one or more processors, a sample tumor fraction, the sample tumor fraction corresponding to a highest tumor fraction from the plurality of tumor fractions;
   determining, using the one or more processors, a plurality of clonal fractions associated with the plurality of somatic alterations, a clonal fraction of the plurality of clonal fractions based on the sample tumor fraction and a tumor fraction for a particular somatic alteration of the plurality of somatic alterations; and
   classifying, using the one or more processors, the sample as clonal with respect to the particular somatic alteration if the clonal fraction is greater than a threshold.
90. A system comprising:
   one or more processors; and
   a memory communicatively coupled to the one or more processors and configured to store instructions that, when executed by the one or more processors, cause the system to perform a method, the method comprising:
      receiving, using one or more processors, sequence read data associated with the sample from the subject;
      determining, using the one or more processors, at least one somatic alteration based on the sequence read data;
      determining, using the one or more processors, a tumor fraction for each somatic alteration of the at least one somatic alteration based on the sequence read data, such that a plurality of tumor fractions is obtained;
      determining, using the one or more processors, a sample tumor fraction, the sample tumor fraction corresponding to a highest tumor fraction from the tumor fraction for each somatic alteration of the at least one somatic alterations; and
      determining, using the one or more processors, a clonal fraction of a somatic alteration of the at least one somatic alteration based on the sample tumor fraction and the corresponding tumor fraction of the somatic alteration.
91. A non-transitory computer-readable storage medium storing one or more programs, the one or more programs comprising instructions, which when executed by one or more processors of a system, cause the system to perform a method comprising:
   receiving, using one or more processors, sequence read data associated with the sample from the subject;
   determining, using the one or more processors, at least one somatic alteration based on the sequence read data;
   determining, using the one or more processors, a tumor fraction for each somatic alteration of the at least one somatic alteration based on the sequence read data, such that a plurality of tumor fractions is obtained;
   determining, using the one or more processors, a sample tumor fraction, the sample tumor fraction corresponding to a highest tumor fraction from the tumor fraction for each somatic alteration of the at least one somatic alterations; and
   determining, using the one or more processors, a clonal fraction of a somatic alteration of the at least one somatic alteration based on the sample tumor fraction and the corresponding tumor fraction of the somatic alteration.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
determining, using the one or more processors, at least one somatic alteration based on the sequence read data;
determining, using the one or more processors, a tumor fraction for each somatic alteration from the at least one somatic alteration, such that at least one tumor fraction is obtained;
identifying, using the one or more processors, a tumor fraction with a highest value from the at least one tumor fraction as a sample tumor fraction; and
determining, using the one or more processors, a clonal fraction for each somatic alteration from the at least one somatic alteration based on (1) the sample tumor fraction, and (2) a corresponding tumor fraction for each somatic alteration.

2. The method of claim 1, wherein the corresponding tumor fraction of the somatic alteration is determined based on an allelic frequency of the somatic alteration, a mutant copy value of the somatic alteration, and a wildtype copy value of the somatic alteration.

3. (canceled)

4. The method of claim 1, further comprising classifying, using the one or more processors, the somatic alteration as clonal if the clonal fraction is greater than a threshold.

5. The method of claim 1, further comprising classifying, using the one or more processors, the somatic alteration as subclonal if the clonal fraction is less than the threshold.

6. (canceled)

7. The method of claim 1, further comprising in accordance with a determination that the clonal fraction is associated with a subclonal alteration, assigning, using the one or more processors, a therapy for the subject based on the clonal fraction.

8. The method of claim 1, further comprising in accordance with a determination that the clonal fraction is associated with a subclonal alteration, administering, using the one or more processors, a treatment to the subject based on the clonal fraction.

9. (canceled)

10. The method of claim 1, further comprising in accordance with a determination that the clonal fraction is associated with a clonal alteration, assigning, using the one or more processors, a treatment to the subject based on the clonal fraction, wherein the therapy comprises a therapy configured to target the clonal alteration.

11. The method of claim 1, further comprising determining, using the one or more processors, a prognosis of the subject based on the clonal fraction.

12. The method of claim 1, further comprising monitoring, using the one or more processors, a progression of a disease of the subject based on the clonal fraction.

13. (canceled)

14. The method of claim 1, further comprising in accordance with a determination that the clonal fraction is associated with a clonal alteration, identifying the respective alteration as a driver of disease in the subject.

15. The method of claim 1, further comprising predicting, using the one or more processors, one or more clinical outcomes based on the clonal fraction.

16. The method of claim 1, wherein the sequence read data for the subject is based on one or more of a broad panel sequencing panel, a targeted-exome sequencing panel, or a whole exome sequencing technique.

17. The method of claim 1, further comprising in accordance with a determination that the clonal fraction is associated with a subclonal alteration, recommending chemotherapy as a treatment.

18. The method of claim 1, wherein the sequence read data for the subject is derived from multiple biopsy samples or a single biopsy sample.

19. The method of claim 1, wherein the sequence read data for the subject is derived from single cell sequencing.

20. The method of claim 1, wherein the sequence read data for the subject is derived from circulating tumor DNA in a liquid biopsy sample.

21. The method of claim 1, wherein the determination of the clonal fraction is used to diagnose or confirm a diagnosis of disease in the subject.

22. The method of claim 1, wherein the disease is cancer.

23. The method of claim 1, further comprising selecting an anti-cancer therapy to administer to the subject based on the determination of the clonal fraction.

24. (canceled)

25. The method of claim 1, wherein the determination of the clonal fraction associated with the sample is used in: (i) making suggested treatment decisions for the subject, and/or (ii) applying or administering a treatment to the subject.

26. (canceled)

27. A method comprising:
providing a plurality of nucleic acid molecules obtained from a sample from a subject;
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules;
sequencing, by a sequencer, the captured nucleic acid molecules to obtain a plurality of sequence reads that represent the captured nucleic acid molecules;
receiving, at one or more processors, sequence read data for the plurality of sequence reads;
determining, using the one or more processors, a quality control metric of the sample;
determining, using the one or more processors, a plurality of tumor fraction estimates for each of a plurality of somatic alterations identified in the sample based on the sequence read data;
determining, using the one or more processors, a sample tumor fraction, the sample tumor fraction corresponding to a highest tumor fraction estimate from the plurality of tumor fraction estimates;
determining, using the one or more processors, a plurality of clonal fractions each clonal fraction corresponding to each of the plurality of somatic alterations; and
classifying, using the one or more processors, the sample as clonal with respect to a particular somatic alteration of the plurality of somatic alterations if a corresponding clonal fraction is greater than a threshold.

28. A method for determining a clonality of alterations in a sample, the method comprising:
receiving, at one or more processors, sequence read data for a plurality of sequence reads derived from the sample;
determining, using the one or more processors, a plurality of tumor fractions for each of a plurality of somatic alterations based on the sequence read data;
determining, using the one or more processors, a sample tumor fraction, the sample tumor fraction corresponding to a highest tumor fraction from the plurality of tumor fractions;
determining, using the one or more processors, a plurality of clonal fractions associated with the plurality of somatic alterations, a clonal fraction of the plurality of clonal fractions based on the sample tumor fraction and a tumor fraction for a particular somatic alteration of the plurality of somatic alterations; and
classifying, using the one or more processors, the sample as clonal with respect to the particular somatic alteration if the clonal fraction is greater than a threshold.
